SEQUENCE LISTING (66 sequences)

SEQ ID NO: 1 - DNA, 672 bp, Saccharomyces cerevisiae
  modified_base (186)..(186): silent point mutation introduced to generate HindIII restriction site
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcacggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaa

SEQ ID NO: 2 - protein, 224 aa, Saccharomyces cerevisiae <s300> NCBI / NC_001136 2004-08-30
MSVINFTGSSGPLVKVCGLQSTEAAECALDSDADLLGIICVPNRKRTIDPVIARKISSLVKAYKNSSGTPKYLVGVFRNQPKEDVLALVNDYGIDIVQLHGDESWQEYQEFLGLPVIKRLVFPKDCNILLSAASQKPHSFIPLFDSEAGGTGELLDWNSISDWVGRQESPESLHFMLAGGLTPENVGDALRLNGVIGVDVSGGVETNGVKDSNKIANFVKNAKK

SEQ ID NO: 3 - DNA, 132 bp, Saccharomyces cerevisiae
  modified_base (22)..(22): point mutation
atgtctgttattaatttcacatgtagttctggtccattggtgaaagtttgcggcttgcagagcacagaggccgcagaatgtgctctagattccgatgctgacttgctgggtattatatgtgtgcccaataga

SEQ ID NO: 4 - protein, 44 aa, Saccharomyces cerevisiae
MSVINFTCSSGPLVKVCGLQSTEAAECALDSDADLLGIICVPNR

SEQ ID NO: 5 - DNA, 540 bp, Saccharomyces cerevisiae
aagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcacggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaa

SEQ ID NO: 6 - protein, 180 aa, Saccharomyces cerevisiae
KRTIDPVIARKISSLVKAYKNSSGTPKYLVGVFRNQPKEDVLALVNDYGIDIVQLHGDESWQEYQEFLGLPVIKRLVFPKDCNILLSAASQKPHSFIPLFDSEAGGTGELLDWNSISDWVGRQESPESLHFMLAGGLTPENVGDALRLNGVIGVDVSGGVETNGVKDSNKIANFVKNAKK

SEQ ID NO: 7 - DNA, 159 bp, Saccharomyces cerevisiae
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgca

SEQ ID NO: 8 - protein, 53 aa, Saccharomyces cerevisiae
MSVINFTGSSGPLVKVCGLQSTEAAECALDSDADLLGIICVPNRKRTIDPVIA

SEQ ID NO: 9 - DNA, 516 bp, Saccharomyces cerevisiae
gcaaggaaaatttcaagtcttgtaaaagcttataaaaatagttcaggcactccgaaatacttggttggcgtgtttcgtaatcaacctaaggaggatgttttggctctggtcaatgattacggcattgatatcgtccaactgcacggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaa

SEQ ID NO: 10 - protein, 172 aa, Saccharomyces cerevisiae
ARKISSLVKAYKNSSGTPKYLVGVFRNQPKEDVLALVNDYGIDIVQLHGDESWQEYQEFLGLPVIKRLVFPKDCNILLSAASQKPHSFIPLFDSEAGGTGELLDWNSISDWVGRQESPESLHFMLAGGLTPENVGDALRLNGVIGVDVSGGVETNGVKDSNKIANFVKNAKK

SEQ ID NO: 11 - DNA, 561 bp, Saccharomyces cerevisiae
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcttataaaaatagttcaggcactccgaaatacttggttggcgtgtttcgtaatcaacctaaggaggatgttttggctctggtcaatgattacggcattgatatcgtccaactgcacggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggt

SEQ ID NO: 12 - protein, 187 aa, Saccharomyces cerevisiae
MSVINFTGSSGPLVKVCGLQSTEAAECALDSDADLLGIICVPNRKRTIDPVIARKISSLVKAYKNSSGTPKYLVGVFRNQPKEDVLALVNDYGIDIVQLHGDESWQEYQEFLGLPVIKRLVFPKDCNILLSAASQKPHSFIPLFDSEAGGTGELLDWNSISDWVGRQESPESLHFMLAGGLTPENVG

SEQ ID NO: 13 - DNA, 111 bp, Saccharomyces cerevisiae
gatgcgcttagattaaatggcgttattggtgttgatgtaagcggaggtgtggagacaaatggtgtaaaagactctaacaaaatagcaaatttcgtcaaaaatgctaagaaa

SEQ ID NO: 14 - protein, 37 aa, Saccharomyces cerevisiae
DALRLNGVIGVDVSGGVETNGVKDSNKIANFVKNAKK

SEQ ID NO: 15 - DNA, 612 bp, Saccharomyces cerevisiae
  mutation (22)..(22): point mutation
atgtctgttattaatttcacatgtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcttataaaaatagttcaggcactccgaaatacttggttggcgtgtttcgtaatcaacctaaggaggatgttttggctctggtcaatgattacggcattgatatcgtccaactgcacggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtg

SEQ ID NO: 16 - protein, 204 aa, Saccharomyces cerevisiae
MSVINFTCSSGPLVKVCGLQSTEAAECALDSDADLLGIICVPNRKRTIDPVIARKISSLVKAYKNSSGTPKYLVGVFRNQPKEDVLALVNDYGIDIVQLHGDESWQEYQEFLGLPVIKRLVFPKDCNILLSAASQKPHSFIPLFDSEAGGTGELLDWNSISDWVGRQESPESLHFMLAGGLTPENVGDALRLNGVIGVDVSGGV

SEQ ID NO: 17 - DNA, 36 bp, Saccharomyces cerevisiae
  mutation (1)..(1): missing sequence before base 1 of SEQ17, corresponding to base 637 of wild-type GAGACAAATGGTGTAAAAGACTCT
aacaaaatagcaaatttcgtcaaaaatgctaagaaa

SEQ ID NO: 18 - protein, 12 aa, Saccharomyces cerevisiae
NKIANFVKNAKK

SEQ ID NO: 19 - protein, 43 aa, Artificial Sequence
  first of a pair of peptides (together with peptide C2), that associate into an anti-parallel coiled coil (Biochemistry 37 (1998),12603-12610)
MDYKDESGQALEKELAQNEWELQALEKELAQLEKELQAGSGSG

SEQ ID NO: 20 - protein, 50 aa, Artificial Sequence
  second of a pair of peptides (together with peptide C1), that associate into an anti-parallel coiled coil (Biochemistry 37 (1998),12603-12610)
GGSGSGQALKKKLAQLKWKLQALKKKNAQLKKKLQAGSYPYDVPDYAAFL

SEQ ID NO: 21 - DNA, 39 bp, Artificial Sequence
  Primer for Ctrp fusions
cgatacgaattcatggacaaggattgtgaaatgaaacgc

SEQ ID NO: 22 - DNA, 45 bp, Artificial Sequence
  Primer for pRS316-C1/2CUP1
aaaggaattggcccaaaatgagtgggagttacaagcacttgagaa

SEQ ID NO: 23 - DNA, 45 bp, Artificial Sequence
  Primer for pRS316-C1/2CUP1
ctcaatgttcgtgaactcttcctcgagcgagttgaactcttcctc

SEQ ID NO: 24 - DNA, 45 bp, Artificial Sequence
  Primer for pRS316-C1/2CUP1
ctccttctcaagttgagcgagctccttctcaagtgcttgtaactc

SEQ ID NO: 25 - DNA, 42 bp, Artificial Sequence
  Primer for pRS316-C1/2CUP1
ggcacttaagaagaagttggcgcagcttaagtggaaactgca

SEQ ID NO: 26 - DNA, 46 bp, Artificial Sequence
  Primer for pRS316-C1/2CUP1
agctgggcattcttcttcttaagagcttgcagtttccacttaagct

SEQ ID NO: 27 - DNA, 48 bp, Artificial Sequence
  Primer for pRS316-C1/2CUP1
aagaagaagaatgcccagcttaagaagaagctccaggctggaagttac

SEQ ID NO: 28 - DNA, 45 bp, Artificial Sequence
  Primer for pRS316-C1/2CUP1
atacgatgttccagattacgctgcatttttataagtcgactggtc

SEQ ID NO: 29 - DNA, 22 bp, Artificial Sequence
  Primer for pRS316-C1/2CUP1
gaccagtcgacttataaaaatg

SEQ ID NO: 30 - DNA, 25 bp, Artificial Sequence
  Primer for circular permutation of TRP1
gtaaaagcttataaaaatagttcag

SEQ ID NO: 31 - DNA, 36 bp, Artificial Sequence
  Primer for circular permutation of TRP1
gaaatagcctaggatgtctgttattaatttcacagg

SEQ ID NO: 32 - DNA, 35 bp, Artificial Sequence
  Primer for circular permutation of TRP1
cagacatcctaggctatttcttagcatttttgacg

SEQ ID NO: 33 - DNA, 25 bp, Artificial Sequence
  Primer for circular permutation of TRP1
tttataagcttttacaagacttgaa

SEQ ID NO: 34 - DNA, 22 bp, Artificial Sequence
  Primer for pRS316-C1/2CUP1
gtaacgaattcatggactacaa

SEQ ID NO: 35 - DNA, 66 bp, Artificial Sequence
  Primer for pRS316-C1/2CUP1
cattttgggccaattccttttccagtgcttgaccactttcgtctttgtagtccatgaattcgttac

SEQ ID NO: 36 - DNA, 64 bp, Artificial Sequence
  Primer for pRS316-C1/2CUP1
caacttcttcttaagtgcctgacctgatccagatccaccgttaacacctgaacctgatccggcc

SEQ ID NO: 37 - DNA, 43 bp, Artificial Sequence
  Primer for pRS316-C1/2CUP1
gtaatctggaacatcgtatgggtaacttccagcctggagcttc

SEQ ID NO: 38 - DNA, 66 bp, Artificial Sequence
  Primer for homologous recombination
gactctaacaaaatagcaaatttcgtcaaaaatgctaagaaatagagggccgcatcatgtaattag

SEQ ID NO: 39 - DNA, 65 bp, Artificial Sequence
  Primer for homologous recombination
aactttcaccaatggaccagaactacctgtgaaattaataacagacattttgagatccgggtttt

SEQ ID NO: 40 - DNA, 40 bp, Artificial Sequence
  Primer for Ctrp fusions
gcctgatccagatccgccttctggtgattcatcatcttca

SEQ ID NO: 41 - DNA, 40 bp, Artificial Sequence
  Primer for Ctrp fusions
ggcggatctggatcaggcaagagaacaattgacccggtta

SEQ ID NO: 42 - DNA, 40 bp, Artificial Sequence
  Primer for Ctrp fusions
gcctgatccagatccgcctataaaaggtattccgacacca

SEQ ID NO: 43 - DNA, 39 bp, Artificial Sequence
  Primer for Ntrp fusions
gcctgatccagatccgcctgcaataaccgggtcaattgt

SEQ ID NO: 44 - DNA, 40 bp, Artificial Sequence
  Primer for Ctrp fusions
ggcggatctggatcaggcgcaaggaaaatttcaagtcttg

SEQ ID NO: 45 - DNA, 42 bp, Artificial Sequence
  Primer for Ntrp fusions
gcctgatccagatccgccaccaacattttctggcgtcagtcc

SEQ ID NO: 46 - DNA, 39 bp, Artificial Sequence
  Primer for Ctrp fusions
ggcggatctggatcaggcgatgcgcttagattaaatggc

SEQ ID NO: 47 - DNA, 39 bp, Artificial Sequence
  Primer for Ntrp fusions
gcctgatccagatccgcccacacctccgcttacatcaac

SEQ ID NO: 48 - DNA, 28 bp, Artificial Sequence
  Primer for Ctrp fusions
ggcggatctggatcaggcaacaaaatag

SEQ ID NO: 49 - DNA, 25 bp, Artificial Sequence
  Primer for pRS315CUP1/ pRS316CUP1
agcaggatcccattaccgacatttg

SEQ ID NO: 50 - DNA, 41 bp, Artificial Sequence
  Primer for pRS315CUP1/ pRS316CUP1
cctaggttgagatctcttgaattcgttacagtttgtttttc

SEQ ID NO: 51 - DNA, 43 bp, Artificial Sequence
  Primer for pRS315CUP1/ pRS316CUP1
aattcaagagatctcaacctaggatgacggtgggaggtctata

SEQ ID NO: 52 - DNA, 45 bp, Artificial Sequence
  Primer for pRS315CUP1/ pRS316CUP1
cgattgtcgacggtcgtacgctaacgcttctcgttggggtctttg

SEQ ID NO: 53 - DNA, 36 bp, Artificial Sequence
  Primer for Ctrp fusions
acttgtcgactcagttttgttcggctttttcattga

SEQ ID NO: 54 - DNA, 33 bp, Artificial Sequence
  Primer for Ctrp fusions
agatagatctatgcctacaaattacgagtatga

SEQ ID NO: 55 - DNA, 60 bp, Artificial Sequence
  Primer for Ntrp fusions
agatagatctatgcatcatcatcatcatcacagcagctctgttattaatttcacaggtag

SEQ ID NO: 56 - DNA, 59 bp, Artificial Sequence
  Primer for Ntrp fusions
acctgtcgacctagtgatgatgatgatgatgacttttcttagcatttttgacgaaattt

SEQ ID NO: 57 - DNA, 33 bp, Artificial Sequence
  Primer for Ctrp fusions
agtaagatctatgcaccaagattttcaagaaga

SEQ ID NO: 58 - DNA, 68 bp, Artificial Sequence
  Primer for Ctrp fusions
gtaagtcgacttaagcgtaatctggaacatcgtatgggtaggatttcttagcatttttgacgaaattt

SEQ ID NO: 59 - DNA, 66 bp, Artificial Sequence
  Primer for Ntrp fusions
caagagatctatgtacccatacgatgttccagattacgcttctatgtctgttattaatttcacagg

SEQ ID NO: 60 - DNA, 38 bp, Artificial Sequence
  Primer for Ntrp fusions
ggcggatctggatcaggcatgatcagtctgattgcggc

SEQ ID NO: 61 - DNA, 29 bp, Artificial Sequence
  Primer for Ntrp fusions
cgaggtcgacttaccgccgctccagaatc

SEQ ID NO: 62 - DNA, 30 bp, Artificial Sequence
  Primer for Ctrp fusions
gcctgatccagatccgccgtttcggccagc

SEQ ID NO: 63 - DNA, 34 bp, artificial sequence
  Primer for homologous recombination
gtggcctatgctaatgcagggccgcaaattaaag

SEQ ID NO: 64 - DNA, 32 bp, Artificial sequence
  Primer used for homologous recombination
ccctgcattagcataggccactagtggatctg

SEQ ID NO: 65 - DNA, 41 bp, Artificial Sequence
  Primer used for N-trp cloning, Sec62 cloning
gcctgatccagatccgcctctattgggcacacatataatac

SEQ ID NO: 66 - DNA, 38 bp, Artificial Sequence
  Primer used for N-trp cloning, Sec62 cloning
ggcggatctggatcaggcatggtagccgagcaaacaca

(19) United States
(12) Patent Application Publication
(10) Pub. No.: US 2009/0305286 A1
(43) Pub. Date: Dec. 10, 2009

(54) Method for the Identification of Suitable Fragmentation Sites in a Reporter Protein

(75) Inventors: Petra Tafelmeyer, Paris (FR); Kai Johnsson, Lausanne (CH)

Correspondence Address: SHOEMAKER AND MATTARE, LTD, 10 POST OFFICE ROAD - SUITE 100, SILVER SPRING, MD, 20910, US

(73) Assignee: ECOLE POLYTECHNIQUE FEDERALE DE LAUSANNE, Lausanne (CH)

(21) Appl. No.: 12/482,536

(22) Filed: Jun. 11, 2009

Related U.S. Application Data
(62) Division of application No. 10/575,374, filed on Jan. 3, 2007.
(60) Provisional application No. 60/510,231, filed on Oct. 9, 2003.

(51) Int. Cl. C12Q 1/68

(52) U.S. Cl. 435/6

(57) ABSTRACT

The invention concerns a combinatorial method for the generation of new split-protein sensors, and its application towards the (β/α)₈-barrel enzyme N-(5′-phosphoribosyl)-anthranilate isomerase Trp1p from Saccharomyces cerevisiae is demonstrated. The generated split-Trp protein sensors allow for the detection of protein-protein interactions in the cytosol as well as the membrane by enabling trp1 cells to grow on medium lacking tryptophan. This powerful selection thus complements the repertoire of the currently used split-protein sensors and provides a new tool for high-throughput interaction screening.

This application is a division of copending application Ser. No. 10/575374, filed Apr. 10, 2006, which was the U.S. national stage of international application PCT/EP2004/011289, filed Oct. 8, 2004, which claims benefit of U.S. provisional application 60/510231, filed Oct. 9, 2003.

The present invention is related to the field of methods for detecting the interaction of proteins via the use of fusion proteins, commonly referred to as split-protein sensors or two-hybrid assays.

The introduction of the yeast-two hybrid system by Fields and Song in 1989 was a milestone for the analysis of protein-protein interactions in living cells (cf. U.S. Pat. No. 5,667,973 and Fields, S., and Song, O. (1989), Nature 340, 245-246). However, a major limitation of this classical two-hybrid system lies in its restriction to the detection of those protein-protein interactions that can be reproduced within the nucleus of a yeast cell. To overcome this restriction, an alternative to this two-hybrid method was introduced in 1994 by Johnsson and Varshavsky (cf. WO 95/29195 and Johnsson, N., and Varshavsky, A. (1994), Proc Natl Acad Sci USA 91, 10340-10344). Here, the two interacting proteins are expressed as fusion proteins with an N- and a C-terminal fragment of ubiquitin. Upon interaction of the two proteins a quasi-native ubiquitin is formed and subsequently recognized by ubiquitin-specific proteases, resulting in the cleavage of a reporter protein from the C-terminal fragment of ubiquitin. The split-ubiquitin system allows for the detection of interactions between cytoplasmic as well as membrane proteins. Since the introduction of split-ubiquitin, a variety of other split-protein sensors has been developed, including pairs of fragments of dihydrofolate reductase (DHFR), β-galactosidase, β-lactamase, inteins, green fluorescent protein (GFP), cAMP cyclase, glycinamide ribonucleotide transformylase, aminoglycoside phosphotransferase, hygromycin B phosphotransferase, and luciferase (cf. Remy, I., and Michnick, S. W. (1999), Proc Natl Acad Sci USA 96, 5394-5399; Rossi, F., Charlton, C. A., and Blau, H. M. (1997), Proc Natl Acad Sci U S A 94, 8405-8410; Galarneau, A., Primeau, M., Trudeau, L. E., and Michnick, S. W. (2002), Nat Biotechnol 20, 619-622; Wehrman, T., Kleaveland, B., Her, J. H., Balint, R. F., and Blau, H. M. (2002), Proc Natl Acad Sci USA 99, 3469-3474; Ozawa, T., Nogami, S., Sato, M., Ohya, Y., and Umezawa, Y. (2000), Anal Chem 72, 5151-5157; Ozawa, T., Kaihara, A., Sato, M., Tachihara, K., and Umezawa, Y. (2001), Anal Chem 73, 2516-2521; Ghosh, I., Hamilton, A. D., and Regan, L. (2000), Journal of the American Chemical Society 122, 5658-5659). Among these systems only split-ubiquitin was successfully applied to screen for binding partners. Other sensors were used to monitor the interactions between selected pairs of proteins rather than to find new partners by a random library approach. Robust systems that can be used for identifying interaction partners at any location inside the cell and in different hosts are therefore still needed. Ideally the interaction-induced reassociation of such a split-protein sensor would provide the cell with a growth advantage thus allowing a selection for interacting proteins. However, generating new split-protein sensors is technically demanding as it depends critically on identifying suitable fragments that can reconstitute a native-like and active protein. The chosen fragmentation site has to fulfill at least the following criteria: (i) to yield two fragments that efficiently fold into quasi-native protein only when fused to two interacting proteins; (ii) not to significantly impair the activity of the reconstituted protein; (iii) to yield soluble protein fragments that are not readily degraded in vivo. In previous studies, the challenge of rationally finding such sites has been mostly tackled by trial and error.

It is thus an object of the present invention to overcome the above-mentioned drawbacks of the prior art, i.e. to provide a method for identification of suitable fragmentation sites in a reporter protein especially for use as a split-protein sensor, that is not limited by the above-mentioned drawbacks of rational design, and which especially allows for the identification of suitable fragmentation sites in a reporter protein even in the absence of any structural information such as a crystal structure. Further objects of the invention will become apparent to the person of routine skill in the art in view of the following detailed description of the invention.

This object and yet further objects are achieved inter alia by a method for the identification of suitable fragmentation sites in a reporter protein, and related thereto, recombinant DNA sequences and, encoded thereby, first and complementary second subdomains of a reporter protein, host cell lines transformed with said recombinant DNA sequences, a kit of parts comprising DNA-based expression vectors, a method for detecting an interaction between proteins, a use of random circular permutation and a use of a host cell line allowing for homologous recombination according to the independent claims.

Most biological processes are controlled by protein-protein interactions and split-protein sensors have become one of the few available tools for the characterization and identification of protein-protein interactions in living cells. Here we introduce a generally applicable combinatorial approach for the generation of new split-protein sensors and apply it to the (β/α)₈-barrel enzyme N-(5′-phosphoribosyl)-anthranilate isomerase Trp1p from Saccharomyces cerevisiae (cf. Braus, G. H., Luger, K., Paravicini, G., Schmidheini, T., Kirschner, K., and Hutter, R. (1988), J Biol Chem 263, 7868-7875). These so-called split-Trp protein sensors are capable of monitoring the interactions of pairs of cytosolic and membrane proteins. One of the selected split-Trp pairs (⁴⁴Nₜᵣₚ and ⁴⁴Cₜᵣₚ) was chosen by means of an example and successfully applied to monitor protein-protein interactions both at the membrane as well as in the cytosol of yeast. Its selected fragmentation site would not have been easily predicted by theoretical considerations, thus underlining the power of the evolutionary approach according to the invention. The direct read-out through complementation of tryptophan auxotrophy qualifies the split-Trp system for high-throughput applications in yeast and bacteria. Of course, appropriately engineered trp1-deficient host strains are required for such assays, which are however either readily available or easily to be made by the person of routine skill in the art. In addition, the introduced combinatorial approach allows for generating split-protein sensors of almost any reporter protein, thereby yielding tailor-made sensors for different applications.

Trp1p is a relatively small (25 kD), monomeric protein that catalyzes the isomerization of N-(5′-phosphoribosyl)-anthranilate in the biosynthesis of tryptophan (cf. Eberhard, M., Tsai-Pflugfelder, M., Bolewska, K., Hommel, U., and Kirschner, K. (1995), Biochemistry 34, 5419-5428). The DNA coding sequence of Saccharomyces cerevisiae is given in SEQ ID NO: 1, the corresponding amino acid sequence is given in SEQ ID NO: 2. Creating a pair of Trp1p fragments (split-Trp) that only reconstitute the enzymatic activity when linked to interacting proteins allows monitoring this protein interaction through a simple growth assay: otherwise trp1 yeast strains expressing such a split-Trp fusion pair would not be able to grow on medium lacking tryptophan. As many different trp1 strains exist, the interaction assay could be applied immediately in different genetic backgrounds, adding a further attractive feature to a split-Trp sensor. Trp1p is a well-studied member of the prominent class of proteins that fold into a (β/α)₈-barrel, which is the most commonly occurring fold among enzymes. The herein presented approach of identifying suitable fragmentation sites in a reporter protein is thus very broadly applicable. This folding motive has been previously subjected to circular permutation and has been expressed as two separate fragments that spontaneously associate into a functional enzyme (cf. Luger, K., Hommel, U., Herold, M., Hofsteenge, J., and Kirschner, K. (1989), Science 243, 206-210; Eder, J., and Kirschner, K. (1992), Biochemistry 31, 3617-3625). Furthermore, it has been proposed that the (β/α)₈-barrel evolved by tandem duplication from a (β/α)₄-domain (cf. Rocker, B., Schmidt, S., and Sterner, R. (2002), FEBS Lett 510, 133-135). In addition to any practical applications it would therefore add to our understanding where the (β/α)₈-barrel can be split into two fragments that, in contrast to previously described pairs of fragments, reconstitute quasi-native Trp1p only when fused to interacting proteins.

As used herein, a “reporter protein” is understood as a protein or peptide, which possesses a unique activity in vivo and/or in vitro, and which produces a signal that allows the active protein to be easily discernable even within a complex mixture of other proteins or peptides, especially in vivo. Reporter proteins as understood herein are e.g. (i) proteins which are essentially involved in the biosynthetic pathway of formation of an amino acid or an other essential metabolite that is crucial for the organism to survive on medium lacking the respective amino acid or metabolite; or (ii) proteins which are detectable by a characteristic color assay when, preferably in vivo; etc.

As used herein, a “suitable fragmentation site” is understood as an especially randomly chosen position in the amino acid chain (and/or the corresponding gene sequence, respectively), at which a given reporter protein is fragmented into a first subdomain and a complementary second subdomain (and/or the corresponding first subsequence and the complementary second subsequence, respectively), wherein the fragmentation site is suitable in the sense of the present invention, when it fulfils the following demands: (i) to yield two fragments that efficiently fold into quasi-native protein only when fused to two interacting proteins; (ii) not to significantly impair the activity of a reconstituted protein by bringing the two fragments into close proximity especially in vivo; (iii) to yield soluble protein fragments that are not readily degraded in vivo.

As used herein, the term “detectable”, especially “detectable when active” is understood as follows. Detection in the sense of the present invention includes any direct or indirect method of testing for the presence of a reporter protein, especially when reconstituted by fragments thereof, e.g. by chemical, physical, or visual means. Most preferably, detection is performed by a color assay, e.g. fluorescence, chemiluminescence or the like, (in vivo and/or in vitro) and/or a growth assay (in vivo).

As used herein, a “first subdomain” and a “complementary second subdomain” of a reporter protein are understood as follows. A first subdomain represents a first successional part (either an N-terminal-, C-terminal-, integral part or even a part involving both the N-terminal- and the C-terminal part) of a native reporter protein. A complementary second subdomain represents a complementary second part (either an N-terminal, C-terminal, integral part or even a part involving both the N-terminal- and the C-terminal part). The first subdomain and the complementary second subdomain essentially resemble the wild-type sequence, when viewed together, wherein overlapping sequences between both subdomains, that are present in both the first subdomain and the complementary second subdomain can be tolerated as long as the activity of the enzyme is not significantly negatively affected. Moreover, minor deletions, additions or other alterations to the overall sequence can be tolerated, especially at the N-terminus or the C-terminus, as long as the activity of the reporter protein, either as a whole or when reconstituted by its fragments, is not significantly negatively affected.

As used herein, a “first subsequence” and a “complementary second subsequence” are understood as gene sequences encoding for the above-mentioned first subdomain and complementary second subdomain.

As used herein, a “color assay” is understood as a manually or device-supported detection of a change in optical appearance of a sample comprising the reporter protein, or a reporter protein reconstituted by its fragments, incl. color developments as well in the visible as in the invisible spectrum. Color assays are especially preferred, that can be qualitatively detected by the unaided eye e.g. by coloration of living cells in vivo (colonies on a plate or the like), and that can be additionally quantified in an in vitro assay, e.g. for determining the intensity of an interaction between two proteins.

As used herein, a “growth assay” is understood as an assay, that allows for the growth of a cell, e.g. a colony on a plate, when the reporter protein is present or actively resembled by its fragments, and wherein cells fail to grow, when the reporter protein is not present or actively resembled by its fragments. Most preferably, the growth assay suchlike allows for a simple visual selection of positives.

As used herein, “stringent conditions” for hybridization of DNA are understood as follows. Given a specific DNA sequence, a person of skill in the art would not expect substantial variation among species within the claimed genus due to hybridization under such conditions, thus expecting structurally similar DNA.

The method according to the invention for the identification of suitable fragmentation sites in a reporter protein, wherein the reporter protein is detectable when active, comprises the steps of:
(a) providing a DNA sequence encoding for said reporter protein;
(b) creating a library based on the DNA sequence as defined in (a),
   wherein each individual of said library comprises a randomly created first subsequence of the DNA sequence as defined in (a), encoding for a first subdomain of said reporter protein, and
   wherein each individual of said library comprises a randomly created complementary second subsequence of the DNA sequence as defined in (a), encoding for a complementary second subdomain of said reporter protein;
(c) screening and/or selection for restoration of detectable activity of said reporter protein, when said first subdomain and said complementary second subdomain are brought into close proximity;
(d) identifying said first subdomain and/or said first subsequence, and said complementary second subdomain and/or said complementary second subsequence, that lead to restoration of detectable activity of said reporter protein.

By using a combinatorial library approach, comprising randomly created first subsequences and randomly created complementary second subsequences, the drawbacks of rational design of split-protein sensors are overcome. Most advantageously, even fragmentation sites of proteins encoded by said subsequences may thereby be identified, which would have never been readily predicted by any rational approach. First subsequences and complementary subsequences are ideally suitable in the context of the present invention, when reconstitution of activity of the corresponding reporter protein only occurs to a significant extent at all, when both corresponding subdomains are forced into close spatial proximity, but do not self-assemble in order to reconstitute a detectable amount of an active reporter protein.

DNA sequences of suitable reporter proteins are readily available to the person of routine skill in the art (step (a)), e.g. from the National Center for Biotechnology Information (NCBI), National Library of Medicine, Building 38A, Bethesda, Md. 20894. Genes encoding for reporter proteins may then be amplified e.g. from a suitable host cell by PCR using standard techniques and primers suitably designed based on the known DNA sequence (vide supra), or the gene encoding for a reporter protein may be completely built up from suitably designed oligonucleotides de novo.

DNA manipulating techniques that may be used in step (b) for the creation of a library based on said DNA sequence are readily apparent to the person of routine skill in the art, either. In short, N- and C-terminal domains of the wild-type reporter protein are amplified separately from a suitable source of DNA by standard PCR techniques, and are subsequently recombined using standard overlap extension PCR techniques in order to recombine and thereby re-arrange the wild-type gene, preferably now containing the N- and C-termini of the wild-type gene connected with each other and as an internal part of the sequence, and preferably comprising a unique restriction site at the wild-type N- and C-termini. At the same time, suitable restriction sites may be designed at the newly created N- and C-termini in order to allow for efficient subsequent cloning steps; most preferably, the restriction site is designed for the same restriction enzyme at both the N- and C-terminus. Most preferably, the re-arranged DNA construct is inserted into a high-copy plasmid, the plasmid amplified by standard techniques, and the re-arranged DNA of interest is thereafter cut out of the high-copy plasmid using the restriction sites at the newly created N- and C-termini. The rearranged gene is then incubated with a ligase to yield dimerized, oligomerized and circularized DNA construct. Afterwards, these constructs are digested e.g. with a suitable, random-cut DNAse, and fragments corresponding to the wild-type length are preferably thereafter treated with ligase and polymerase, to repair nicks, gaps and to flush the ends of the fragments of the reporter protein. Afterwards, the DNA fragments corresponding to the wild-type length of the reporter protein's gene are isolated e.g. by standard agarose gel electrophoresis procedures. The resulting fragments are preferably blunt-end cloned into a suitable expression vector, which was cleaved at a unique restriction site (preferably blunt-end). The expression vector is especially designed by standard DNA manipulation techniques to provide a construct after blunt-end cloning, in which one of the artificially generated new N- and C-termini is under the control of a promoter sequence and especially fused to a gene encoding for a tag sequence and a gene encoding for first peptide or protein C1, each preferably via a linker sequence. Moreover, the other terminus, respectively, is especially fused to a gene encoding for a preferably different tag sequence and gene encoding for a second peptide or protein C2. Peptides or proteins C1 and C2 are thereby known to interact with each other in vivo, and may e.g. be leucine zippers. The tag sequences may afterwards advantageously be used for the control of correct expression and stability of fusion proteins. After transformation and amplification in a suitable host such as e.g. E. coli XL1Blue to a typical library size of about 10⁴ to 10⁵ independent clones, the vector is linearized at a restriction site at the wild-type N- and C-termini, and an oligonucleotide is inserted into the resulting gap, which is specifically designed to integrate a terminator for the first domain of said reporter protein and a promoter sequence for the second domain of said reporter protein, by homologous recombination in a suitable host such as yeast according to standard procedures. The oligonucleotide is designed and constructed by standard PCR techniques to provide flanking regions both at the 5′ and 3′ ends of e.g. about 50 bp with the gene of the reporter protein in order to allow for successful homologous recombination. Suchlike, the selection of clones possessing fragmentation sites at or nearby the wild-type N- and C-termini can be suppressed. For selecting thereafter, a marker gene is also provided by the oligonucleotide, e.g. encoding for a protein involved in antibiotic resistance. Successful homologous recombination may thus be easily observed by growth in the presence of the respective antibiotic.

Step (c) is preferably carried out by growing the respective transformants of the library on medium which e.g. lacks a nutrient, e.g. an amino acid, or which provides a substrate for a color reaction. Thus, preferably a growth assay or a color assay is performed, thereby allowing for easy selection of those transformants which lead to a restoration of activity of the reporter protein, which is e.g. essentially involved in the synthesis of said nutrient, e.g. said amino acid, or in said color reaction. Step (c) especially involves the elimination of false positives, i.e. first subdomains and complementary second subdomains, that reconstitute an active reporter enzyme by self-reassembling, i.e. without the need of an outer influence forcing the two domains into close spatial proximity. This can be done e.g. by fusing the respective first and second subdomains of the reporter protein to first and second peptides or proteins, that do not interact with each other, and/or by testing the respective first and second subdomains without any first and second peptides fused thereto at all, and/or by testing constructs lacking the first or the second subdomain, respectively. These assays can be performed by techniques commonly known in the art of e.g. two-hybrid assays.

Identification of suitable subdomains and subsequences, i.e. suitable fragementation sites, can be performed by common DNA- and/or protein sequencing techniques.

According to a preferred embodiment, the reporter protein is detectable in vivo and/or in vitro, both as full length protein and when actively resembled by a first subdomain and a complementary second subdomain, by a means chosen from the group consisting of color assays and growth assays.

Growth assays provide the advantage of a selection step, i.e. only positives grow under the chosen conditions, thus eliminating the need of further screening all individuals of the library. Exemplarily, only positives that comprise a suitable combination of first subdomain and complementary second subdomain grow as colonies on nutrition-specific plates. Color assays, moreover, can be individually designed depending on the specific reporter protein, when this reporter protein is involved naturally in or artificially usable for a color-developing reaction. In some cases, a substrate for such a reporter protein may be incorporated into the growth medium, e.g. the plate, whereupon colored colonies appear due to reconstitution of an active reporter protein by a first subdomain and a complementary second subdomain in vivo. Quanification of such an in vivo color assay may be optionally performed with samples obtained from such colonies. The general procedure of growth assays, color assays and subsequent quantification of the color assay are known in principle from the classical two-hybrid system, cf. eg. U.S. Pat. No. 5,667,973, incorporated herein by reference.

In an especially preferred embodiment, individuals of the library as defined in (b) are either prokaryotic or eukaryotic host cells, comprising:
both said first subsequence and said complementary second subsequence in one and the same expression vector, suitable for (co-)expression of said first subsequence and said complementary second subsequence in vivo; or
said first subsequence in a first expression vector suitable for (co-)expression of said first subsequence, and said complementary second subsequence in a second expression vector suitable for (co-)expression of said complementary second subsequence.

In vivo assays are at least in the first step preferred, e.g. as a growth assay as outlined above. Thus, prokaryotic or eukaryotic host cells are provided, that are manipulated suchlike to allow for the (co-)expression of both the first and the complementary second subdomain of the reporter protein. Depending on the specific application, both subdomains may of course be encoded by one and the same, or by separate vectors. In most cases, encoding by one and the same vector will be favourable. A vast amount of suitable expression vectors for use as a basis in this respect are available to the person of routine skill in the art, e.g. the pRS316-based yeast expression vector (cf. Sikorski, R. S., and Hieter, P. (1989), Genetics 122, 19-27, incorporated herein by reference).

It is especially preferred that the screening for restoration of detectable activity of said reporter protein, when said first subdomain and said complementary second subdomain are brought into close proximity as defined in (c), comprises the following steps:
creating a first fusion subsequence comprising the first subsequence of said reporter protein as defined in (b), fused to an oligonucleotide encoding for a first protein or peptide,
creating a second fusion subsequence comprising the complementary second subsequence of said reporter protein as defined in (b), fused to an oligonucleotide encoding for a second protein or peptide,
   wherein said first protein or peptide and said second protein or peptide are known to interact.

By creating said first fusion sequence and said second fusion subsequence, the first subdomain and the complementary second subdomain are forced into close spatial proximity, thus allowing for a screening for restoration of activity of the reporter protein, when the subdomains are forced into close proximity. Preferably, said first protein or peptide and said second protein or peptide are chosen to be robust and relatively small proteins or peptides; especially preferred in the context of the invention are leucine zippers, most preferably leucine zippers which associate to an anti-parallel coiled coil (interacting proteins fused to 3′-terminus of the first subdomain and the 5′-terminus of the second subdomain, or vice versa, respectively). However, for specific embodiments, a parallel orientation may be preferred, e.g. for testing membrane proteins which most commonly exhibit both the N- and the C-terminus to one and the same site.

According to a further embodiment said first fusion subsequence and said second subsequence are created by blunt end ligation.

Blunt end ligation is the method of choice for the construction of said fusion subsequences, as due to the evolutionary, random approach of library generation no predictable, specific sticky-end ligation can be performed. Although blunt-end ligation leads to the creation of statistical amounts of ligation products which are out of the reading frame, this approach still proved sufficiently efficient for the identification of suitable fragmentation sites according to the invention.

Moreover, in another especially preferred embodiment said first fusion subsequence and said second fusion subsequence each comprise
a linker sequence in between said first subsequence (or said second subsequence, respectively) and said oligonucleotide encoding for a first protein or peptide (or said oligonucleotide encoding for a second protein or peptide, respectively);
at least one tag that allows for verification of the transcription of said first fusion subsequence and said second fusion subsequence.

Linker sequences commonly prove useful in the art of construction of fusion proteins in order to both allow for proper folding of both components of the fusion protein individually or cooperatively, and/or to achieve sufficient spatial integrity of both components of the fusion protein.

The use of tag sequences that allow for the detection of transcription of a gene sequence is also routinely applied in the art. In the context of the present invention, tag sequences may be applied to any of the N- and C-terminus of the first subdomain and/or the N- and C-terminus of the complementary second subdomain. It is especially preferred to provide differently recognizable tag sequences both at the N- and the C-termini of each transcription product. Commonly applied tags are e.g. the HA tag, the flag tag or the like. Detection of correct expression of these tags, and thereby of the fusion protein(s), may be performed e.g. by Western-blotting according to routine procedures.

According to an especially preferred embodiment, an oligonucleotide is inserted by homologous recombination in between said first subsequence and said second subsequence, encoding for:
a transcription terminating sequence for terminating transcription of said first or said second subsequence;
a transcription promoting sequence for initiating transcription of said second or said first subsequence, respectively;
a marker sequence allowing for control of successful homologous recombination.

An especially advantageous way of carrying out the present invention is to simply initially provide said first and said second subsequence continuously, preferably rearranged, and thereafter to separate them by introducing a transcription terminating sequence succeeding the first subsequence, and a transcription promoting sequence preceeding the second subsequence. Thereby, separate expression is secured of both the first subdomain and the complementary second subdomain, or their fusion domains, respectively. This goal may be especially advantageously achieved by homologous recombination at a predefined site in between said first and said second subsequence (c.f. Oldenburg, K. R., Vo, K. T., Michaelis, S., and Paddon, C. (1997), Nucleic Acids Res 25, 451-452, incorporated herein by reference).

In order to eliminate the otherwise high risk of isolating subdomains, that are fragmented at fragmentation sites nearby the N- and C-termini of the wild-type reporter protein, it is especially preferred to not provide the DNA sequence of said reporter protein according to step (a), vide supra, in its wild-type configuration, but rather already with the wild-type N- and C-termini connected with each other and being an internal part of the DNA sequence of said DNA sequence. Thereby, artificial new N- and C-termini are created in the starting material. Most preferably, a unique restriction site RE2 is introduced in between the wild-type N- and C-terminus. A further restriction site RE1 is advantageously introduced at the new artificial N- and C-terminus of the DNA sequence of said reporter protein according to step (a), allowing for easy and convenient cloning and construction of libraries according to step (b), vide supra. Due to the unique restriction site RE2, homologous recombination in a suitable host cell can be performed in between the wild-type N- and C-terminus of the reporter protein. Due to the necessary overlap for successful homologous recombination, isolation of subdomains with fragmentation sites at or nearby the wild-type N- and C-terminus is suppressed. Most preferably, the oligonucleotide used for homologous recombination comprises a selection marker such as e.g. a gene involved in antibiotic resistance in order to check for successful homologous recombination.

Thus, in a further embodiment, the method comprises the steps of:
creating fragmentation sites in TRP1 using gene cleavage with a unique restriction enzyme RE1 and circularization;
isolating fragments corresponding to the wild-type length;
subcloning using blunt ends preferably into a pRS316 based yeast expression vector under the control of a copper promoter (pCUB1) and transforming into E. coli, preferably XL1Blue;
recombining and amplifying homologues with a unique restriction site RE2, preferably AvrII, introduced between the original N- and C-termini to allow subsequent linerization of the vector;
locating two leucine zippers in the plasmid at the 3′- and the 5′-ends of the newly generated N- and C-termini, the zippers being positive and negative charged helices to allow heterodimerization, preferably each heterodimer containing a buried asparagine residue in a position to force antiparallel orientation of the zippers.

The invention further relates to a recombinant DNA sequence for use in securing expression in a prokaryotic or eukaryotic host cell of a polypeptide product having the primary structural conformation of a first subdomain of a reporter protein or a complementary second subdomain of a reporter protein, wherein detectable activity of said reporter protein is restored, when said first subdomain and said complementary second subdomain are brought into close proximity, and wherein said first and said complementary second subdomain are not subdomains of one of the group of proteins consisting of transcriptional activators, ubiquitin, dihydrofolate reductase, β-lactamase, green fluorescent protein and closely related variants such as e.g. ECFP, EGFP or the like, β-galactosidase, inteins, cAMP cyclase, glycinamide ribonucleotide transformylase, aminoglycoside

In the above-mentioned and herewith disclaimed DNA sequences, suitable fragmentation sites for split-protein sensors were already identified by rational design (cf. e.g. Methods Enzymology 238, Michnick et al. 2000). However, the present invention now opens up for the first time the possibility to identify suitable fragmentation sites in any other DNA sequence encoding for a reporter protein by a random library approach, too. Providing this tool to the person of routine skill in the art by the method disclosed herein, suitable fragmentation sites may be now identified with relative ease.

In especially preferred embodiments, said DNA sequence encodes for a subdomain of a (β/α)₈-barrel enzyme, such as e.g. Trp1p.

In further embodiments, which proved especially advantageous, said DNA sequence is selected from the group consisting of:
(a) the DNA sequences set out in Table 1 and their complementary strands;
(b) DNA sequences which hybridize under stringent conditions to the protein coding regions of the DNA sequences defined in (a) or fragments thereof;
(c) DNA sequences which, but for the degeneracy of the genetic code, would hybridize to the DNA sequences defined in (a) or (b) and which sequences code for a polypeptide having the same amino acid sequence.

The above-mentioned DNA sequences encode for the split-Trp sensors split-Trp⁴⁴ (i.e. ⁴⁴Nₜᵣₚ and ⁴⁴Cₜᵣₚ), split-Trp⁵³ (i.e. ⁵³Nₜᵣₚ and ⁵³Cₜᵣₚ) split-Trp¹⁸⁷ (i.e. ¹⁸⁷Nₜᵣₚ and ¹⁸⁷Cₜᵣₚ), split-Trp^{204b} (i.e. ^{204b}Nₜᵣₚ and ^{204b}Cₜᵣₚ), which proved to be valuable tools as split-protein sensors (numbering according to the fragmentation site, given as the last amino acid of the N-terminal subdomain). Especially split-Trp44 was successfully applied herein to demonstrate the interaction of membrane proteins.

The DNA- and amino acid sequences of the above-mentioned split-Trp sensors are given in the attached sequenced listing as follows:

| | |
|---|---|
| SEQ ID NO: 3 | ⁴⁴Nₜᵣₚ (DNA sequence); |
| SEQ ID NO: 4 | ⁴⁴Nₜᵣₚ (amino acid sequence); |
| SEQ ID NO: 5 | ⁴⁴Cₜᵣₚ (DNA sequence); |
| SEQ ID NO: 6 | ⁴⁴Cₜᵣₚ (amino acid sequence); |
| SEQ ID NO: 7 | ⁵³Nₜᵣₚ (DNA sequence); |
| SEQ ID NO: 8 | ⁵³Nₜᵣₚ (amino acid sequence); |
| SEQ ID NO: 9 | ⁵³Cₜᵣₚ (DNA sequence); |
| SEQ ID NO: 10 | ⁵³Cₜᵣₚ (amino acid sequence); |
| SEQ ID NO: 11 | ¹⁸⁷Nₜᵣₚ (DNA sequence); |
| SEQ ID NO: 12 | ¹⁸⁷Nₜᵣₚ (amino acid sequence); |
| SEQ ID NO: 13 | ¹⁸⁷Cₜᵣₚ (DNA sequence); |
| SEQ ID NO: 14 | ¹⁸⁷Cₜᵣₚ (amino acid sequence); |
| SEQ ID NO: 15 | ^{204b}Nₜᵣₚ (DNA sequence); |
| SEQ ID NO: 16 | ^{204b}Nₜᵣₚ (amino acid sequence); |
| SEQ ID NO: 17 | ^{204b}Cₜᵣₚ (DNA sequence); |
| SEQ ID NO: 18 | ^{204b}Cₜᵣₚ (amino acid sequence); |

In preferred embodiments according to the present invention, said DNA sequences are used in securing expression in a prokaryotic or eukaryotic host cell of a polypeptide fusion product. Such securing of expression may be achieved by any means routinely applied by the person of routine skill in the art, comprising e.g. incorporation of said DNA sequences into suitable expression vectors or integration of said DNA sequences into the genome of said host.

The invention further relates to a first subdomain of a reporter protein or a complementary second subdomain of a reporter protein, wherein detectable activity of said reporter protein is restored, when said first subdomain and said complementary second subdomain are brought into close proximity, and wherein said first and said complementary second subdomain are not subdomains of one of the group of proteins consisting of transcriptional activators, ubiquitin, dihydrofolate reductase, β-lactamase, green fluorescent protein and closely related variants such as e.g. ECFP, EGFP or the like, β-galactosidase, inteins, cAMP cyclase, glycinamide ribonucleotide transformylase, aminoglycoside phosphotransferase, hygromycin B phosphotransferase, luciferase.

In the above-mentioned and herewith disclaimed proteins, suitable fragmentation sites for split-protein sensors were already identified by rational design. However, the present invention now opens up for the first time the possibility to identify suitable fragmentation sites in any other reporter protein by a random library approach, too. Providing this tool to the person of routine skill in the art by the method disclosed herein, suitable fragmentation sites may be now identified with relative ease.

According to especially preferred embodiments of the invention, a first subdomain of a reporter protein or a complementary second subdomain of a reporter protein are produced by a method of culturing a host transformed with a recombinant DNA sequence as outlined above, wherein said molecules further comprises an ex-pression control sequence, said expression control sequence being operatively linked to said molecule. Said expression control sequences comprise especially those which are commonly referred to as tags which are recognizable e.g. by Western-blotting procedures routinely applied in the art.

The invention further relates to a fusion protein comprising a first subdomain of a reporter protein or a complementary second subdomain of a reporter protein as outlined above, and a further peptide or protein connected thereto in a naturally not occurring combination. By creating such artificial fusion proteins, said further protein of peptide may then be tested for interaction with e.g. a specifically chosen counterpart or against a library of possible counterparts. Moreover, library-library screening assays may also be applied, e.g. genome-wide library screenings as e.g. already performed in the art of traditional two-hybrid assay.

The invention further relates to a prokaryotic or eukaryotic host cell line, transformed with recombinant DNA sequences as outlined above.

Said prokaryotic or eukaryotic host cell lines are preferably E. coli or yeast strains. For cloning and storage purposes, mostly E. coli strains such as XL1Blue will be chosen. For the method of identification of suitable fragmentation sites according to the invention, especially involving the step of homologous recombination, a yeast strain may be chosen such as e.g. Saccharomyces cerevisiae, e.g. EGY48, and Schizosaccharomyces pombe. The choice of a suitable host cell line is routinely performed by the person of skill in the art, depending on the specific purpose; such host cell lines are commonly available.

The invention is further related to a kit of parts, comprising a first and a second DNA-based expression vector, wherein
said first expression vector contains an expression cassette encoding for a polypeptide product having at least a substantial part of the primary structural confirmation of a first subdomain of a reporter protein; and
said second expression vector contains an expression cassette encoding for a polypeptide product having at least a substantial part of the primary structural confirmation of a complementary second subdomain of a reporter protein; and wherein detectable activity of said reporter protein is restored, when said first subdomain and said complementary second subdomain are brought into close proximity, and wherein said first and said complementary second subdomain are not subdomains of one the group of proteins consisting of transcriptional activators, ubiquitin, dihydrofolate reductase, β-lactamase, green fluorescent protein and closely related variants such as e.g. ECFP, EGFP or the like, β-galactosidase, inteins, cAMP cyclase, glycinamide ribonucleotide transformylase, aminoglycoside phosphotransferase, hygromycin B phosphotransferase, luciferase.

According to a further especially preferred embodiment, such a kit of parts further comprising a suitable prokaryotic or eukaryotic host cell line for expression of said first and second expression vector.

Having provided by the present invention a tool for identifying novel fragmentation sites in reporter proteins, another major aspect of the present invention is related to a method for detecting an interaction between a first test peptide or protein or a fragment thereof, and a second test peptide or protein or a fragment thereof, the method comprising the steps of:
providing recombinant DNA sequences as outlined above for use in securing expression of a first subdomain of a reporter protein and a complementary second subdomain of a reporter protein;
fusing an oligonucleotide or a gene encoding for a first test peptide or protein to the DNA sequence encoding for said first subdomain of the reporter protein, thereby creating a first DNA fusion sequence encoding for a fusion protein comprising said first subdomain of the reporter protein and said first test peptide or protein;
fusing an oligonucleotide or a gene encoding for a second test peptide or protein to the DNA sequence encoding for said complementary second subdomain of the reporter protein, thereby creating a second DNA fusion sequence encoding for a fusion protein comprising said complementary second subdomain of the reporter protein and said second test peptide or protein;
(co-)expressing said fusion protein comprising said first subdomain of the reporter protein and said first test peptide or protein, and said fusion protein comprising said second complementary subdomain of the reporter protein and said second test peptide or protein in a suitable prokaryotic or eukaryotic host cell;
screening and/or selecting for restoration of detectable activity of said reporter protein.

Utilizing split-protein sensors with subdomains identified by a method according to the invention, interaction of said first test peptide and said second test peptide may be identified. Given the tool of identifying suitable fragmentation sites in virtually any reporter protein, the person of routine skill in the art is no more hampered by the limitations of the existing, rationally designed split-protein systems to specific cellular compartments, but rather may now choose a reporter protein depending on his specific test purpose.

In the most preferred embodiment, a library of oligonucleotides or DNA encoding for a set of first test peptides or proteins and/or a library of oligonucleotides or DNA encoding for a set of second test peptides or proteins are fused to said first subdomain of said reporter protein and/or said complementary second subdomain of said reporter protein, respectively.

According to an especially preferred embodiment of the present invention, the interaction between a first test peptide or protein or a fragment thereof and a second test peptide or protein or fragment thereof is mediated by a chemical inducer of dimerization, which binds either covalently or non-covalently to both said test peptides or proteins or fragments thereof.

Comparable systems are commonly referred to in the literature as three-hybrid systems. Chemical inducers of dimerization (CIDs) have been first described by Schreiber and Crabtree (c.f. Spencer D. M, Wandless T. J, Schreiber S. L, and Crabtree G. R (1993), Science 262, 1019-1024, incorporated herein by reference). CIDs are cell-permeable molecules that can simultaneously form a covalent- or non-covalent interaction with two different proteins or peptides, thereby inducing their dimerization. Using split-protein sensors according to the present invention, e.g. robust drug and/or drug target screening assays may easily be established. Towards this aim, e.g. Nₜᵣₚ may be fused to a protein library and Cₜᵣₚ to an O(6)-alkylguanine-DNA alkyltransferase (AGT), e.g. human AGT (hAGT). A substrate for hAGT, e.g. Benzylguanine, may be easily covalently linked to a multitude of small molecules (hypothetical drugs), thus allowing for an efficient screening for cellular targets contained in said protein library that react or associate with the corresponding drug.

Moreover, the invention is related to a method for detecting the interruption of an interaction between a first test peptide or protein or a fragment thereof, and a second test peptide or protein or a fragment thereof, the method comprising the steps of:
providing recombinant DNA sequences according to one of claims 11 to 14 for use in securing expression of a first subdomain of a reporter protein and a complementary second subdomain of a reporter protein;
fusing an oligonucleotide or a gene encoding for a first test peptide or protein to the DNA sequence encoding for said first subdomain of the reporter protein, thereby creating a first DNA fusion sequence encoding for a fusion protein comprising said first subdomain of the reporter protein and said first test peptide or protein;
fusing an oligonucleotide or a gene encoding for a second test peptide or protein to the DNA sequence encoding for said complementary second subdomain of the reporter protein, thereby creating a second DNA fusion sequence encoding for a fusion protein comprising said complementary second subdomain of the reporter protein and said second test peptide or protein;
(co-)expressing said fusion protein comprising said first subdomain of the reporter protein and said first test peptide or protein, and said fusion protein comprising said second complementary subdomain of the reporter protein and said second test peptide or protein in a suitable prokaryotic or eukaryotic host cell;
screening and/or selecting for interruption of interaction of said first subdomain and said second subdomain under the influence of one or more test agents.

Comparable systems are commonly referred to in the literature as reverse two-hybrid systems (or split-protein systems, respectively). Exemplarily, 5-fluoroanthranilic acid (FAA) is metabolized in vivo into a toxic product by the tryptophan biosynthetic enzymes. Applying the split-Trp sensors according to the invention, the disruption of protein-protein interaction leading to the spatial separation of the Trp1p fragments (and thus inactivity of the reporter protein) can therefore be linked to the survival of the cells on medium containing FAA. By means of example, libraries of small molecules may be screened for their ability to interact with a pair of fusion proteins. Selection of proteins or peptides that disrupt an interaction can be done by co-expressing two interacting proteins with a random protein or peptide library e.g. on plates containing FAA. The reverse split-Trp sensors may also advantageously be used to determine the binding region of a protein. A random library of the protein carrying mutations is co-expressed with its binding partner on plates containing FAA. Only cells that express a library member with mutations in or affecting the binding region, thus disrupting the interaction of the two proteins, will be able to grow in the presence of FAA.

Another aspect of the present invention is related to a use of random circular permutation of a gene and/or the expressed polypeptide derived thereof for the identification of fragmentation sites in a reporter protein for use in a split-protein sensor. To date, random circular permutation has not been used for the identification of such suitable fragmentation sites for separately expressed subdomains, but rather for the identification of proteins of at least approximately wild-type length, but with artificially new N- and C-termini, and with the wild-type N- and C-termini being connected to each other and being an internal part of the sequence. However, this approach now surprisingly proved to be an outstandingly valuable tool for the evolutionary, combinatorial approach of identifying suitable fragmentation sites for subdomains to be expressed separately.

A further aspect of the present invention is related to a use of a host cell line that allows for homologous recombination of DNA for the generation of a recombinant DNA molecule that secures for expression of both a polypeptide product comprising a first subdomain of a reporter protein and a complementary second subdomain of a reporter protein from said recombinant DNA molecule. To date, homologous recombination has not been used for this purpose, but has now surprisingly found to be an outstandingly valuable tool for simply and conveniently securing for expression of a first subdomain and a complementary second subdomain of a reporter protein.

### DETAILED DESCRIPTION OF THE INVENTION

The invention will now be described in even more detail by means of an example and a specific embodiment, together with the accompanying figures; however, without the invention being limited thereto.

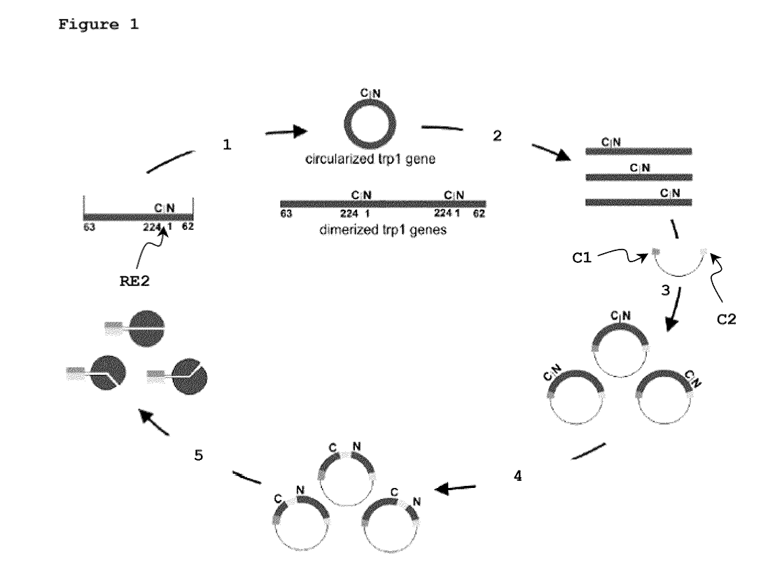

FIG. 1: Combinatorial approach towards the generation of split-Trp sensors. As a starting point, a rearranged copy of the TRP1 gene was used in which the original N- and C-termini of TRP1 were connected by a short linker encoding a unique restriction site RE2, here an AvrII site. For convenient subcloning, another restriction site RE1 was introduced at the artificially created new N- and C-termini, here a HindIII site. The linear fragment was incubated with T4 DNA ligase to circularize/oligomerize the gene (step 1). Treatment of the ligation mix with DNAseI resulted in randomly cut linear molecules and fragments corresponding to the size of TRP1 were isolated (step 2). Isolated fragments were cloned into a yeast expression vector containing two polypeptides (C1 and C2) that associate into an antiparallel-coiled coil (step 3). Homologous recombination in yeast cells was used to insert a terminator sequence and the P_{GAL1}-promoter between the original N- and C-termini (step 4). Co-expression of the two fragments and selection for complementation of tryptophan auxotrophy of yeast cells allowed the isolation of functional split-Trp pairs.

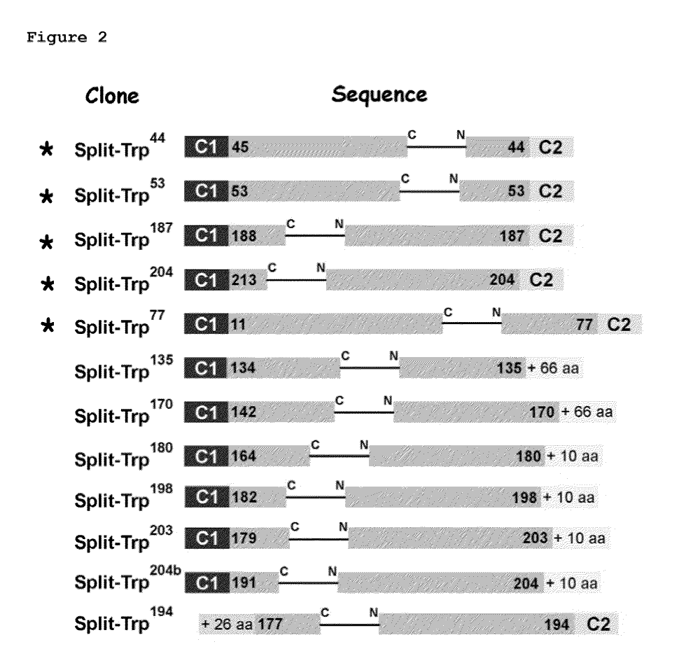

FIG. 2: Selected split-Trp protein pairs capable of complementing tryptophan auxotrophy in yeast. The clones are named after the last residue of each N-terminal fragment. C1 and C2 are the two polypeptides that associate into the anti-parallel coiled coil. Due to a shift in the reading frame in 5 of the twelve clones, C2 is replaced by peptide of 10 or 66 amino acids, and C1 is replaced in one clone by a peptide of 26 residues. Five of the twelve analyzed clones lead to the expression of Trp1p fragments in which both fragments were fused in frame to the polypeptides C1 and C2 (marked with an asterisk).

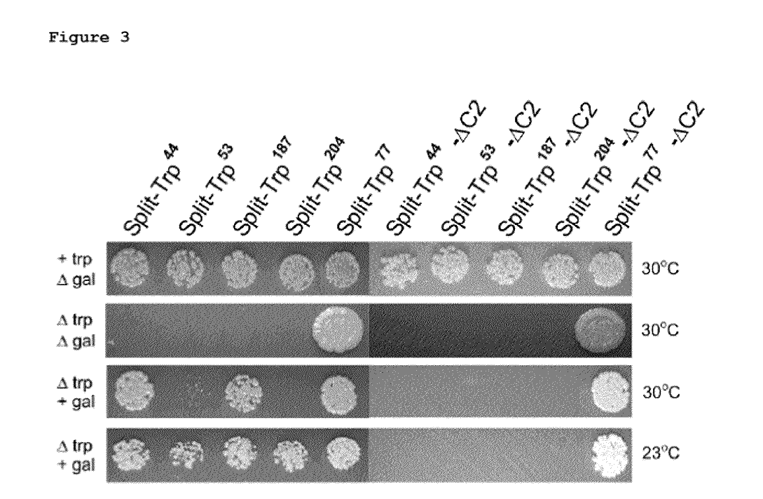

FIG. 3: Characterization of the selected split-Trp pairs that are marked with an asterisk in FIG. 2. Growth assays of yeast strains expressing split-Trp⁴⁴, split-Trp⁵³ split-Trp¹⁸⁷, split-Trp^{204b} or split-Trp⁷⁷ on selective plates (+/Δ trp: plates with tryptophan/lacking tryptophan, respectively; +/Δ gal: plates with galactose/lacking galactose). For control experiments, yeast strains expressing the split-Trp proteins in which the sequence encoding for C2 was deleted form the plasmid (split-Trp-ΔC2) were also investigated. One colony of yeast cells EGY48 expressing different split-Trp protein pairs was resuspended in 1 ml water and 5pl were spotted on medium with or without tryptophan and/or galactose, but always containing copper at two different temperatures (30° C. and 23° C.). C1-Cₜᵣₚ is under control of the leaky P_{CUP1}-promoter and Nₜᵣₚ-C2 under the control of the P_{GAL1}-promoter. Images were taken after 8 days.

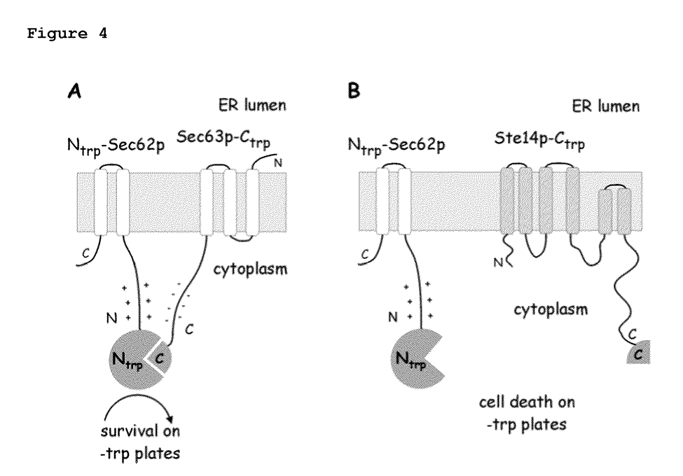

FIG. 4: Analysis of the interaction between Sec62p and Sec63p using the split-Trp system. Left: Nₜᵣₚ is fused to the N terminus of Sec62p and Cₜᵣₚ is fused to the C terminus of Sec63p, resulting in Nₜᵣₚ-Sec62p and Sec63p-Cₜᵣₚ, respectively. The linker between the cytosolic domains of Sec62p and Sec63p and the corresponding Trp1p fragments consists of six residues. The known interaction between the positively charged cytosolic N-terminal domain of Sec62p and the negatively charged C-terminal tail of Sec63p should lead to the reconstitution of active Trp1p and complementation of tryptophan auxotrophy. Right: Co-expression of Nₜᵣₚ-Sec62p with Ste14p-Cₜᵣₚ, a further membrane protein of the ER, which does not interact with Sec62p, should not lead to the formation of a functional Trp1p and the complementation of tryptophan auxotrophy.

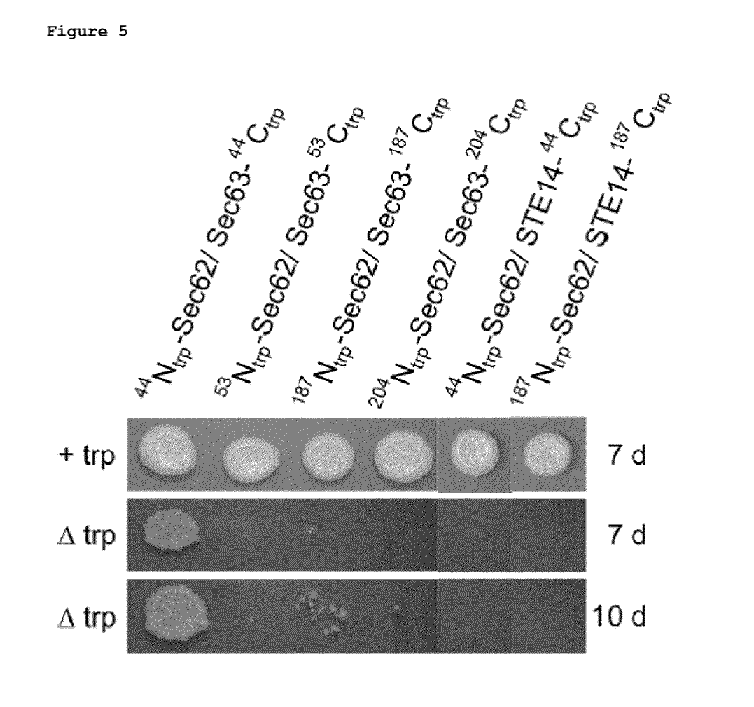

FIG. 5: Split-Trp interaction assay of Sec62p and Sec63p. A colony of EGY48 cells co-expressing Nₜᵣₚ-Sec62p with Sec63p-Cₜᵣₚ or Ste14p-Cₜᵣₚ was suspended in 1 ml water and 5 μl were spotted on copper containing medium with or without tryptophan. Cells co-expressing ⁴⁴Nₜᵣₚ-Sec62p/Sec63p-⁴⁴Cₜᵣₚ complement tryptophan auxotrophy as indicated by their growth after 4 days at 23° C. Large colonies were visible after 7 days of incubation, whereas only small colonies were observed for cells expressing ¹⁸⁷Nₜᵣₚ-Sec62p/Sec63p-¹⁸⁷Cₜᵣₚ. No or only very small colonies were observed for cells co-expressing ⁵³Nₜᵣₚ-Sec62p/Sec63p-⁵³Cₜᵣₚ or ^{204b}Nₜᵣₚ-Sec62p/Sec63p-^{204b}Cₜᵣₚ, respectively. No growth was observed for cells co-expressing ⁴⁴Nₜᵣₚ-Sec62p/Ste14p-⁴⁴Cₜᵣₚ or ¹⁸⁷Nₜᵣₚ-Sec62p/Ste14p-¹⁸⁷Cₜᵣₚ even after 10 days of incubation at 23° C.

DNA- and protein sequences SEQ ID NO: 1 to SEQ ID NO: 66, as given in the attached sequence listing, are given in the attached sequence listing, incl. all primers and oligonucleotides used for the construction of the vectors.

For any standard molecular biology and especially DNA- and protein manipulation protocols it is generally referred to Sambrook, J. et al., eds., MOLECULAR CLONING, A LABORATORY MANUAL, 2nd. edition, Cold Spring Harbor Laboratory Press, Cold Spring Harbor, N.Y. (1989); Ausubel, F. et al., eds., CURRENT PROTOCOLS IN MOLECULAR BIOLOGY, John H. Wiley & Sons, Inc. (1997); HYBRID HUNTER™ INSTRUCTION MANUAL, Invitrogen BV, Groningen, Neherlands (1999); Burke, D et al., METHODS IN YEAST GENETICS. A COLD SPRING HARBOR LABORATORY COURSE MANUAL, Cold Spring Harbor Laboratory Press (2000).

Yeast Media. Yeast complete medium containing adenine (YPAD) was used for cultures of Saccharomyces cerevisiae EGY48 and RSY529. Dropout media (YC) were used to select for the presence of pRS315- or pRS316-derived plasmids and for the complementation of tryptophan auxotrophy. Lacking amino acids or components in the resulting medium are indicated by the addition of their one-letter code to the YC-dropout medium. Selective YC-medium used to plate out the yeast cells after transformation by electroporation was supplemented with 1 M sorbitol. For the expression of proteins from the P_{GAL1}-promoter 2% galactose and 0.5% raffinose replaced glucose as carbon source in the YC-medium.
YPAD: 1% yeast extract, 2% peptone, 2% dextrose, 100 mg/l adenine, (2% agar for plates)
YC 0.12% yeast nitrogen base, 0.5% ammonium sulfate, 1% succinic acid, 2% glucose, 0.6% NaOH, 1.4 g/l yeast synthetic dropout medium omitting histidine (H), leucine (L), tryptophan (W) and uracil (U), (2% agar for plates),
-L: 0.05 g/l histidine (H), 0.1 g/l tryptophan (W), 0.1 g/l uracil (U)
-U: 0.05 g/l histidine (H), 0.1 g/l leucine (L), 0.1 g/l tryptophan (W)
-LU: 0.05 g/l histidine (H), 0.1 g/l tryptophan (W)
-LUW: 0.05 g/l histidine (H)

Transformation of yeast cells. The transformation of Saccharomyces cerevisiae strains EGY48 or RSY529 with one or more plasmids was done using a standard protocol for transformation by electroporation. An overnight culture of EGY48 or RSY529 yeast cells in YPAD medium was diluted in 500 ml YPAD to an OD₆₀₀ of ˜0.3 and grown at 30° C. and 260 rpm to an OD₆₀₀ of ˜1.4. The culture was harvested by centrifugation at 4300 rpm and washed with 500 ml and 250 ml ice-cold sterile water and with 30 ml ice-cold 1 M sorbitol. The pelleted cells were then resuspended in 300-500 μl 1 M sorbitol and either used directly for transformation or frozen in aliquots of 40 μl at −80° C. For the double transformation of two plasmids, competent cells were always prepared freshly. A total amount of 100 ng plasmid DNA was mixed with 40 μl competent yeast cells, and electroporated at 1.5 kV using a Stratagene electroporator 1000 in a 0.2 mm cuvette. The cells were mixed with 500 μl ice-cold 1 M sorbitol immediately after the pulse and plated on the corresponding solid selective YC-medium containing 1 M sorbitol.

Cloning of pRS316-C1/2_{CUP1}. A sequence containing two polypeptides C1 and C2 was first assembled by PCR using a set of primers as described by Stemmer et al (cf. Oakley M. G., and Kim P. S. (1998), Biochemistry 37, 12603-12610; Oakley M. G, and Hollenbeck J. J. (2001), Curr Opin Struct Biol 11, 450-457; Stemmer W. P., Crameri A., Ha K. D., Brennan T. M., Heynecker H. L. (1995), Gene 164, 49-53; all incorporated herein by reference). In short, the primers were mixed in an equimolar concentration (12.5 μM of each primer) and assembled in 55 cycles of denaturation (94° C., 30 s), primer annealing (52° C., 30 s) and extension (72° C., 30 s) using 0.1 unit/μl Pwo polymerase and 0.5 mM of each dNTP in the gene assembly buffer (10 mM Tris-HCl, pH 8.8, 2.2 mM MgCl₂, 50 mM KCl and 0.1% Triton X-100). The double gene was then amplified out of this reaction using Pwo polymerase with the 5′-primer PTP116 that contains an EcoRI site and the 3′-primer PTP111 that contains a SalI site. The PCR product was cleaved with EcoRI and SalI and cloned into pRS316, resulting in pRS316-C1/2 (cf. Sikorski R. S, and Hieter, P. (1989), Genetics 122, 19-27, incorporated herein by reference). The final construct contained the sequences for an N-terminal FLAG tag, the polypeptide C1 followed by a five-residue linker, an HpaI blunt end restriction site and a six-residue-linker followed by the polypeptide C2 with a C-terminal HA tag. C1 and C2 are two peptides that associate into an antiparallel-coiled coil (cf. Oakley M. G., and Kim P. S. (1998), Biochemistry 37, 12603-12610; Oakley M. G, and Hollenbeck J. J. (2001), Curr Opin Struct Biol 11, 450-457). The sequence of the P_{CUP1}-promoter was then cleaved out of the plasmid pAGTM2-Dha with BamHI and EcoRI and positioned upstream of the C1/C2 cassette in pRS316-C1/2, resulting in pRS316-C1/2_{CUP1}.

Cloning of pRS315_{CUP1} and of pRS316_{CUP1}. The pRS315-derived vector was constructed for an easy cloning of the different Nₜᵣₚ-SEC62 constructs, whereas the pRS316-derived vector was constructed for an easy cloning of the different SEC63-Cₜᵣₚ constructs (cf. Sikorski R. S, and Hieter, P. (1989), Genetics 122, 19-27, incorporated herein by reference). The sequence of the P_{CUP1}-promoter of the plasmid pAGTM2-Dha was amplified by PCR with the primers PTP181 and PTP182. The gene of ECFP was amplified by PCR out of pLP-ECFP-C1 with the primers PTP183 and PTP184. Both fragments were then combined by overlap extension PCR using the 5′-primer PTP181 that contains a BamHI site and the 3′-primer PTP184 that contains a SalI site, so that the P_{CUP1}-promoter is upstream of ECFP (cf. Ho S. N. et al. (1989), Gene 1989, 51-59; incorporated herein by reference). The partially homologous primers PTP182 and PTP183 contain the sequence of the restriction sites EcoRI, BglII and AvrII to allow a versatile cloning of genes downstream of the P_{CUP1}-promoter. The final fragment consisting of P_{CUP1}-promoter and ECFP was then cloned into pRS315 or pRS316 with BamHI and SalI, resulting in pRS315_{CUP1} or pRS316_{CUP1} (cf. Sikorski R. S, and Hieter, P. (1989), Genetics 122, 19-27).

To generate split-protein sensors based on Trp1p (split-Trp) we adapted an approach originally developed by Graf and Schachmann for creating random circular permutations of proteins (cf. Graf, R., and Schachman, H. K. (1996), Proc Natl Acad Sci USA 93, 11591-11596, incorporated herein by reference). Using PCR, the TRP1 gene of Saccharomyces cerevisiae was first rearranged so that it started with residue 63 and its former start codon was fused to the stop codon via a linker sequence encoding a unique AvrII restriction site. The N- and the C-terminal domains of TRP1 were therefore amplified separately out of the plasmid pYESTrp2 (Invitrogen) with the primers PTP113/115 and PTP112/114, respectively, and recombined using overlap extension PCR with the primers PTP112 and PTP115 (cf. Ho S. N. et al. (1989), Gene 1989, 51-59; incorporated herein by reference). This rearrangement was performed to avoid unwanted isolation of wild-type gene in the subsequent selections. At the same time, a HindIII restriction site was introduced via the PCR primers at the newly generated N- and C-termini by introducing a silent mutation in the gene at around amino acid 63. Since the direct digestion of PCR products in former experiments yielded a product that did not ligate efficiently, the rearranged gene was first inserted into a high-copy plasmid (pAK400) and, after amplification of the vector DNA, cut out with HindIII. The rearranged gene was then incubated with T4 DNA ligase at 16° C. for 14 h at a DNA concentration of 0.14 mg/ml, leading to the formation of circular DNA as well as dimers and higher oligomers. After inhibition of the ligase at 65° C. for 20 min and desalting of the solution using a microcon PCR column, the ligation products were incubated with DNaseI (˜1.2 units/mg DNA) in 50 mM Tris-HCl, pH 7.5, 1 mM MnCl₂ at 25° C. for six minutes. The exact conditions for the DNaseI reactions were determined immediately before the digestion in small test reactions. The DNaseI reaction was stopped by phenol extraction and ethanol precipitation. After incubation of the digested DNA with T4 DNA ligase and T4 polymerase to repair nicks, gaps and to flush the ends of the fragments, DNA fragments corresponding to the size of the original gene were isolated by gel electrophoresis. These fragments were ligated into the pRS316-based yeast expression vector pRS316-C1/2_{CUP1} that was cleaved with HpaI and dephosphorylated according to standard protocols. In the resulting vector, the C-terminal half of TRP1 is fused to a gene encoding for a FLAG tag, a polypeptide C1 and a five-residue linker sequence and is expressed under the control of the P_{CUP1}-promoter. The N-terminal half of TRP1 is fused to a gene encoding for a six-residue linker sequence, the polypeptide C2 and a HA tag. The sequences of the peptides C1 and C2, including epitope tag and linker are:

(SEQ ID NO: 19)

C1: MDYKDESGQALEKELAQNEWELQALEKELAQLEKELQ
AGSGSG,

(SEQ ID NO: 20)

C2: GGSGSGQALKKKLAQLKWKLQALKKKNAQLKKKLQAG
SYPYDVPDYAAFL,

After transformation in XLlBlue, resulting in a library with about 3×10⁴ independent clones, the bacteria were scratched from the plate, and the plasmids isolated and linearized with AvrII. To insert a terminator for the C-terminal fragment and a promoter for the N-terminal fragment, a DNA fragment was constructed by PCR consisting of the CYC1-terminator, a geneticin resistance gene, the P_{GAL1}-promoter and flanking regions of about 50 base pairs at the 5′-and 3′-ends homologous to the original N and C termini of Trp1p. The CYC1-terminator was amplified out of pYESTrp2 with the primers PTP107 and PTP120, whereas the cassette containing the geneticin resistance gene and the P_{GAL1}-promoter was amplified out of pFA6a-GAL1 with the primers PTP108 and PTP121. Both fragments were combined by overlap extension PCR using the primers PTP120 and PTP121 (cf. Ho S. N. et al. (1989), Gene 1989, 51-59; incorporated herein by reference). The linearized vector (0.3 μg) and the PCR fragment (3 μg) were then co-transformed in chemically competent EGY48 cells and plated on plates lacking uracil but containing geneticin (500 μg/ml) to select for insertion of the PCR fragment into the linearized vector through homologous recombination (cf. Oldenburg et al. (1997), Nucleic Acids Res 25, 451-452;. incorporated herein by reference). Chemically competent yeast cells were prepared as described by standard protocols. The homologous recombination also suppressed the predominant isolation of TRP1 genes that were cut near the original N or C terminus. In the final construct, the C-terminal fragment fused to C1 (C1-Cₜᵣₚ) is under the control of the inducible but leaky P_{CUP1}-promoter and the N-terminal fragment fused to C2 (Nₜᵣₚ-C2) is under the stringent control of the P_{GAL1}-promoter. After 3 days of incubation at 30° C., approximately 1600 colonies were isolated and subsequently replica-plated on plates lacking uracil and tryptophan but containing geneticin (250 μg/ml), galactose (2%) and CuSO₄ (0.1 mM). After replica plating, 45 colonies were able to complement tryptophan auxotrophy. Approximately half of those 45 colonies required the presence of galactose and CuSO₄ to grow on plates lacking tryptophan and twelve of these clones were then analyzed by DNA sequencing (FIG. 2). Five of the twelve analyzed clones lead to the expression of Trp1p fragments in which both fragments were fused in frame to the polypeptides C1 and C2 (marked with an asterisk in FIG. 2). Seven of the twelve clones were out of frame with C1 or C2. These frame shifts resulted in the replacement of C2 in split-Trp¹³⁵ and split-Trp¹⁷⁰ with a peptide of 66 residues possessing the sequence DLDQVRHLRRSWRSLSGNCKLLRRRMPSLRRSSRLEVTHMFQITLHFYKSTSRGGPVPSFCSL and in split-Trp¹⁸⁰, split-Trp¹⁹⁸, split-Trp²⁰³ and split-Trp^{204b} with a peptide of 10 residues possessing the sequence (E/Q)RWIWIRSGT. It is assumed that Nₜᵣₚ and Cₜᵣₚ of these clones associate spontaneously without the help of interacting proteins. In split-Trp⁴⁴ and split-Trp^{204b} the mutation Gly8Cys was introduced during the fragmentation procedure. However, the influence of this mutation seems to be of minor importance as the deletion of the first ten amino acids still allowed split-Trp⁷⁷ to complement tryptophan auxotrophy (FIGS. 2 and 3).

For split-Trp⁴⁴, split-Trp⁵³, split-Trp¹⁸⁷, split-Trp^{204b} and split-Trp⁷⁷ the sequence encoding Nₜᵣₚ-C2 was deleted from the plasmid using BglII and SalI and replaced with a PCR fragment encoding only the corresponding Nₜᵣₚ fragment. The resulting constructs were then retransformed into EGY48 (FIG. 3). To test whether the trp1 complementation depends on the presence of both Trp1p fragments we repeated the growth assays on plates lacking tryptophan and galactose but containing glucose and copper, thereby repressing the expression of Nₜᵣₚ-C2. Of the five clones tested, only split-Trp⁷⁷ conferred tryptophan auxotrophy to the trp1 yeast in the presence of glucose by itself, indicating that its large C-terminal fragment spanning residues 11-224 already possesses enzymatic activity. On galactose, split-Trp⁴⁴ split-Trp¹⁸⁷ and split-Trp⁷⁷ complemented tryptophan auxotrophy at 30° C. and 23° C., whereas split-Trp⁵³ and split-Trp^{204b} complemented tryptophan auxotrophy only at 23° C. (FIG. 3).

The deletion of C2 abolished the capacity of the four clones split-Trp⁴⁴, split-Trp⁵³, split-Trp187, split-Trp^{204b} to complement tryptophan auxotrophy (FIG. 3). This finding demonstrates that the formation of a functional Trp1p from these fragments indeed depends on the fusion to a pair of interacting polypeptides.

Since the structure of Trp1p from S. cerevisiae has not yet been solved, we aligned its sequence with the sequences of the N-(5′-phosphoribosyl)-anthranilate isomerases from E. coli (ePRAI) and Thermotoga maritima (tPRAI), and identified the fragmentation sites in the known crystal structures of the homologous enzymes (FIG. 4). The fragmentation site of split-Trp⁴⁴ lies in one of the active site loops between β2 and α2, two residues away from an arginine residue that interacts with the carboxyl group of the substrate N-(5′-phosphoribosyl)-anthranilate. Although combinatorial mutagenesis experiments have indicated that turn sequences in general are highly mutable in (β/α)₈-barrels, the vicinity of this position to an active site residue would not have made it an obvious candidate for a fragmentation site (cf. Silverman, J. A., Balakrishnan, R., and Harbury, P. B. (2001), Proc Natl Acad Sci USA 98, 3092-3097). In split-Trp¹⁸⁷ and split-Trp⁵³ the fragmentation sites are located in α-helices α7 and α2 of the (β/α)₈-barrel, respectively. This appears plausible in hindsight with the mutability of α-helical residues in combinatorial mutagenesis experiments on (β/α)₈-barrels and with earlier random circular permutation experiments of other folds in which new termini were introduced into α-helices (cf. Silverman, J. A., Balakrishnan, R., and Harbury, P. B. (2001), Proc Natl Acad Sci USA 98, 3092-3097; Graf, R., and Schachman, H. K. (1996), Proc Natl Acad Sci USA 93, 11591-11596). Furthermore, α-helix α2 is extended by nine amino acids in Trp1p compared to ePRAI and tPRAI, making it plausible that the introduction of a fragmentation site could be tolerated without significantly affecting the activity or the folding of the (β/α)₈-barrel. Particularly interesting is split-Trp^{204b}, in which a stretch of eight amino acids (205-212), including four highly conserved residues, is deleted from Trp1p. This results in a very short Cₜᵣₚ of only twelve residues that is fused to C1, corresponding to α-helix α8 in the structure of tPRAI and ePRAI. The eight deleted amino acids form a loop in the vicinity of the active site, directly after the short α-helix α8′. Helix α8′ is believed to participate in the binding of the phosphate group of the substrate and is not present in the regular structures of other (β/α)₈-barrels (cf. Eder, J., and Kirschner, K. (1992), Biochemistry 31, 3617-3625; Hennig, M., Sterner, R., Kirschner, K., and Jansonius, J. N. (1997), Biochemistry 36, 6009-6016). While split-Trp^{204b} complements tryptophan auxotrophy only at 23° C., indicating a decreased stability of the split enzyme, this finding nevertheless questions the significance of this loop with its four completely conserved residues in the function of N-(5′-phopsphoribosyl)-anthranilate isomerases. However, it is unknown how much residual Trp1p activity is sufficient to complement tryptophan auxotrophy in yeast and a more detailed interpretation of this finding will therefore require the kinetic characterization of Split-Trp^{204b} in in vitro assays. Eder and Kirschner have shown that the N-terminal fragment 1-167 folds in the absence of its C-terminal partner (cf. Eder, J., and Kirschner, K. (1992), Biochemistry 31, 3617-3625). Furthermore, it has been proposed that this N-terminal subdomain is an intermediate in the folding of Trp1p (cf. Silverman, J. A., Balakrishnan, R., and Harbury, P. B. (2001), Proc Natl Acad Sci USA 98, 3092-3097; Kirschner, K., Szadkowski, H., Henschen, A., and Lottspeich, F. (1980), J Mol Biol 143, 395-409; Jasanoff, A., Davis, B., and Fersht, A. R. (1994), Biochemistry 33, 6350-6355; Silverman, J. A., and Harbury, P. B. (2002), J Mol Biol 324, 1031-1040; Sanchez del Pino, M. M., and Fersht, A. R. (1997), Biochemistry 36, 5560-5565). In agreement with these studies all of the selected split-Trp pairs that spontaneously assemble into a functional protein possess relatively large N-terminal fragments, incorporating at least the first five (β/α)-motives. This observation suggests that a spontaneous assembly of Trp1p fragments depends on the presence of a folded N-terminal domain and that the location of the fragmentation site reflects the folding pathway of the natural protein. Shorter N-terminal fragments such as ⁴⁴Nₜᵣₚ and ⁵³Nₜᵣₚ might not fold independently and the chances to spontaneously reconstitute active protein from unfolded fragments without induced proximity would be greatly diminished. Noteworthy, most of the isolated split-Trp pairs that reassemble spontaneously consist of Trp1p fragments that overlap for at least 13 residues. This overlap prevents us to exactly localize the fragmentation site from the sequence data (FIG. 2). An exception is split-Trp¹³⁵ where, according to the structure of tPRAI, the fragmentation site is located in a loop at the N-terminal side of the (β/α)₈-barrel.

### Detection of Membrane Protein Interactions Using Split-Trp Sensors

An important application for new split-protein sensors will lie in the detection and characterization of protein-protein interactions occurring at the membranes of intracellular organelles and the cell membranes. To test whether the split-Trp system operates at the membrane, the interaction-dependent split-Trp pairs were attached to the membrane proteins Sec62p and Sec63p (FIG. 4) (cf. Panzner, S., Dreier, L., Hartmann, E., Kostka, S., and Rapoport, T. A. (1995), Cell 81, 561-570; Deshaies, R. J., and Schekman, R. (1989), J Cell Biol 109, 2653-2664; Wittke, S., Dunnwaid, M., and Johnsson, N. (2000), Mol Biol Cell 11, 3859-3871). Sec62p and Sec63p directly bind to each other and are part of the heptameric Sec-complex that is responsible for translocating proteins posttranslationally across the membrane of the endoplasmic reticulum (ER) (FIG. 5A). Briefly, SEC62 was fused to the 3′-end of the N-terminal fragment of the four split-Trp systems, allowing for the expression of ⁴⁴Nₜᵣₚ-Sec62p, ⁵³Nₜᵣₚ-Sec62p, ¹⁸⁷Nₜᵣₚ-Sec62p and ^{204b}Nₜᵣₚ-Sec62p. SEC63 was fused to the 5′-end of the corresponding C-terminal fragments, allowing for the expression of Sec63p-⁴⁴Cₜᵣₚ, Sec63p-⁵³Cₜᵣₚ, Sec63p-¹⁸⁷Cₜᵣₚ and Sec63p-^{204b}Cₜᵣₚ.

To monitor the interaction between Sec62p and Sec63p, trp1 yeast strains expressing pairs of matching Nₜᵣₚ-Sec62p and Sec⁶³p-Cₜᵣₚ fusion proteins were spotted on selective plates lacking tryptophan (FIG. 5). Strains co-expressing 44Nₜᵣₚ-Sec62p/Sec63p-⁴⁴Cₜᵣₚ, ¹⁸⁷Nₜᵣₚ-Sec62p/Sec63p-¹⁸⁷Cₜᵣₚ and ^{204b}Nₜᵣₚ-Sec62p/Sec63p-^{204b}Cₜᵣₚ were able to grow on plates lacking tryptophan at 23° C. but not at 30° C. Only small colonies were detected after 7 days for ¹⁸⁷Nₜᵣₚ-Sec62p/Sec⁶³p-¹⁸⁷Cₜᵣₚ and after 10 days for ^{204b}Nₜᵣₚ-Sec62p/Sec63p-^{204b}Cₜᵣₚ, whereas strains co-expressing ⁴⁴Nₜᵣₚ-Sec62p/Sec63p-⁴⁴Cₜᵣₚ grew significantly faster. No growth at all was observed for strains expressing ⁵³Nₜᵣₚ-Sec62p/Sec63p-⁵³Cₜᵣₚ. To verify that the observed complementation of tryptophan auxotrophy is a result of the interaction between the Sec62p and Sec63p moieties of the fusion proteins, we fused the C-terminal fragments of split-Trp⁴⁴ and split-Trp¹⁸⁷ to the cytoplasmic site of Ste14p (FIG. 4B). Ste14p is a membrane protein of the ER that is known to interact with neither Sec62p nor Sec63p (FIG. 4B) (cf. Wittke, S., Lewke, N., Muller, S., and Johnsson, N. (1999), Mol Biol Cell 10, 2519-2530). No growth on plates lacking tryptophan was observed when matching pairs of Sec62p and Ste14p fusion proteins were co-expressed at 23° C. or 30° C. for 10 days (FIG. 5). The cellular amount of Ste14p-⁴⁴Cₜᵣₚ is roughly 2-3 fold lower than the amount of Sec63p-⁴⁴Cₜᵣₚ as determined by western blotting (data not shown). Since this relatively small effect cannot account for the clear growth difference between the strains expressing either ⁴⁴Nₜᵣₚ-Sec62p/Sec⁶³p-⁴⁴Nₜᵣₚ or ⁴⁴Nₜᵣₚ-Sec62p/Ste14p-⁴⁴Cₜᵣₚ, we conclude that the ⁴⁴Nₜᵣₚ-Sec62p/Sec⁶³p-⁴⁴Cₜᵣₚ interaction signal is specific.

In more detail, the gene of SEC62 was amplified by PCR from yeast EGY48 genomic DNA and combined by overlap extension PCR with the N-terminal fragments of split-Trp⁴⁴, split-Trp⁵³, split-Trp¹⁸⁷ and split-Trp^{204b}, yielding ⁴⁴Nₜᵣₚ-SEC62, ⁵³Nₜᵣₚ-SEC62, ¹⁸⁷Nₜᵣₚ-SEC62 and ^{204b}Nₜᵣₚ-SEC62. At the same time, a 6×His tag was introduced at the 5′-end of Nₜᵣₚ. The Nₜᵣₚ genes and SEC62 are connected by a sequence coding for a six-residue linker (GGSGSG). The four Nₜᵣₚ-SEC62 PCR products were isolated by gel electrophoresis and ligated in a pRS315-derived expression vector (LEU2) (pRS³¹⁵_{CUP1}) under the control of the P_{CUP1}-promoter. Towards this aim, the vector was cleaved with BglII and SalI and the ECFP gene was replaced by the corresponding Nₜᵣₚ-SEC62 construct.

The genes of SEC63 and STE14 were amplified by PCR from yeast EGY48 genomic DNA and combined by overlap extension PCR with the C-terminal fragments of split-Trp⁴⁴, split-Trp⁵³, split-Trp¹⁸⁷ and split-Trp^{204b}. At the same time, a 6×His tag was introduced at the 3′-end of Cₜᵣₚ, yielding SEC63-⁴⁴Cₜᵣₚ-His, SEC63-⁵³Cₜᵣₚ-His, SEC63-¹⁸⁷Cₜᵣₚ-His, SEC63-^{204b}Cₜᵣₚ-His, STE14-⁴⁴Cₜᵣₚ-His and STE14-¹⁸⁷Cₜᵣₚ-His. SEC63 and the Cₜᵣₚ-His genes are connected by a sequence coding for a six-residue linker (GGSGSG). The different SEC63-Cₜᵣₚ-His and STE14-Cₜᵣₚ-His PCR products were isolated by gel electrophoresis and ligated into a pRS316-derived vector (URA3) (pRS316_{CUP1}, vide supra) under the control of the P_{CUP1}-promoter. To replace the 6×His tag by the more sensitive HA tag the genes of the different SEC63-Cₜᵣₚ-His and STE14-Cₜᵣₚ-His constructs were amplified by PCR with a 3′-primer that contains an HA tag and cloned into pRS316_{CUP1}. All SEC63 and STE14 fusions contained an HA tag fused to the C terminus of Trp1p. The vector was cleaved with BglII and SalI and the ECFP gene was replaced with the corresponding SEC63-Cₜᵣₚ and STE14-Cₜᵣₚ constructs. All constructs were verified by DNA sequencing.

Expression of Nₜᵣₚ-Sec62p fusion proteins. Expression and functionality of the Nₜᵣₚ-Sec62p fusion proteins was confirmed by complementation of the temperature-sensitive yeast strain RSY529 (MATα his4 leu2-3, 112 ura3-52 sec62-1) (cf. Rothblatt J. A. et al. (1989), J Cell Biol 109, 2641-2652). RSY529 contains an endogenous temperature-sensitive variant of Sec62p. A colony of RSY529 cells transformed with either pRS315 or a pRS315-derived vector expressing ⁴⁴Nₜᵣₚ-Sec62p, ⁵³Nₜᵣₚ-Sec62p, ¹⁸⁷Nₜᵣₚ-Sec⁶²p or ^{204b}Nₜᵣₚ-Sec62p was resuspended in 1 ml water and 5 μl were spotted on YC-L medium containing 0.1 mM CuSO₄ to induce the expression of the fusion proteins and incubated at 30° C. and 38° C. for 6 d to control for the complementation of the temperature sensitivity of RSY529.

Expression of Sec63p-Cₜᵣₚ and Ste14p-Cₜᵣₚ fusion proteins. The expression of the different Sec63p-Cₜᵣₚ and Ste14p-Cₜᵣₚ fusion proteins was verified by immunoblotting using antibodies against the HA tag at the C terminus of Trp1p. Towards this aim, an overnight culture of yeast EGY48 cells containing one of the Sec⁶³p-Ctₜᵣₚ or Ste¹⁴p-Cₜᵣₚ fusion proteins was diluted in 10 ml selective medium YC-U to an OD₆₀₀˜0.8 and grown for 3 h at 30° C. and 220 rpm. Protein expression was induced by adding CuSO₄ to a final concentration of 0.1 mM. After 3 h of expression at 30° C. and 220 rpm, the cell solution (same volume at same OD when different samples were compared) was centrifuged at 4300 rpm for 10 minutes and the pellet resuspended in 150 μl yeast lysis buffer (50 mM HEPES, pH 7.5, 150 mM NaCl, 5 mM EDTA, 1% Triton X-100) containing 1% (v/v) protease inhibitor cocktail and 0.5 mM PMSF. 200 μl glass beads were added and the solution was vortexed at full speed for 3×30 s and cooled on ice in between the vortexing steps. The glass beads and the cell debris were pelleted by centrifugation for 30 s at 13000 rpm and the supernatant was mixed with an appropriate volume of 5×SDS sample buffer (50% glycerol, 7.5% SDS, 250 mM Tris-HCl, pH 8.0, 0.5% Bromphenol blue, 12.5 mM 2-Mercaptoethanol). Proteins were denatured for 3 min at 95° C. Aliquots were analysed by Western blotting (12% SDS-PAGE) as described by standard protocols. After blotting, the nitrocellulose membrane was incubated with 3% dry milk in TBST (10 mM Tris-HCl, 150 mM NaCl, pH 7.9, 0.05% Tween 20) to block unspecific antibody binding. Expression of Sec63p-Cₜᵣₚ or Ste14p-Cₜᵣₚ fusion constructs was controlled by incubation of the membrane with the primary anti-HA antibody 1:7500 in TBST (10 mM Tris-HCl, 150 mM NaCl, pH 7.9, 0.05% Tween 20). An anti mouse-HRP antibody conjugate was used 1:7500 in TBST (10 mM Tris-HCl, 150 mM NaCl, pH 7.9, 0.05% Tween 20) as secondary antibody. Detection was done on a Kodak Image Station 440CF using the NEN Renaissance kit, a luminol-based chemiluminescence system.

The present data demonstrate that in particular split-Trp⁴⁴ is well suited for the detection of protein-protein interactions between membrane proteins. Interestingly, yeast cells co-expressing ⁴⁴Nₜᵣₚ-Sec62p and Sec⁶³p-⁴⁴Cₜᵣₚ require lower growth temperatures for the complementation of tryptophan auxotrophy than the cells expressing the corresponding C1 and C2 coiled coil fusions. This effect might be due to a more favorable orientation of the N- and C-terminal Trp1p fragments in the antiparallel-coiled coil than in the Sec62p/Sec63p complex.

In conclusion, we have used directed evolution to convert N-(5′-phosphoribosyl)-anthranilate isomerase into a split-protein sensor. In coupling the interaction of cytosolic and membrane proteins to a simple growth assay, the split-Trp system possesses all the necessary features to complement already existing systems to measure and screen for new protein interactions. This split-Trp approach may be used in identifying partners of medically relevant targets, e.g. in three-hybrid assays and protein/small molecule interaction assays. Furthermore, the evolutionary approach introduced here is generally applicable to other enzymes. By generating novel split-protein sensors that are based on proteins functioning in the matrix of e.g. the mitochondrium, the peroxisome or the lumen of the secretory path, this evolutionary approach will help to overcome the lack of techniques to measure protein interactions in the interior of these organelles. Finally, the analysis of the different split-Trp pairs that either spontaneously assemble into a functional (β/α)₈-barrel or need to be fused to interacting proteins to yield folded protein supports the hypothesis that a large N-terminal subdomain of Trp1p is an important intermediate in the folding of the (β/α)₈-barrel.

### Further Experimental Details

For the various PCR- and gene assembly reactions, if not already noted explicitly above, the following primers and templates were used.

Primers used for Nₜᵣₚ-constructs (cf. attached sequence listing for details):

| | | | | cloning |
|---|---|---|---|---|
| construct | 5′-primer | 3′-primer | template(s) | sites |
| ⁴⁴Nₜᵣₚ | PTP193 | PTP146 | split-Trp⁴⁴ | — |
| | SEQ ID NO: 55 | SEQ ID NO: 65 | | |
| ⁴⁴Nₜᵣₚ-HA | PTP199 | PTP146 | split-Trp⁴⁴ | — |
| | SEQ ID NO: 59 | SEQ ID NO: 65 | | |
| ⁵³Nₜᵣₚ | PTP193 | PTP170 | split-Trp⁵³ | — |
| | SEQ ID NO: 55 | SEQ ID NO: 43 | | |
| ¹⁸⁷Nₜᵣₚ | PTP193 | PTP172 | split-Trp¹⁸⁷ | — |
| | SEQ ID NO: 55 | SEQ ID NO: 45 | | |
| ^{204b}Nₜᵣₚ | PTP193 | PTP174 | split-Trp^{204b} | — |
| | SEQ ID NO: 55 | SEQ ID NO: 47 | | |
| SEC62 | PTP147 | PTP188 | EGY48 yeast | — |
| | SEQ ID NO: 66 | SEQ ID NO: 53 | genomic DNA | |
| ⁴⁴Nₜᵣₚ-SEC62 | PTP193 | PTP188 | ⁴⁴Nₜᵣₚ/SEC62, | BglII/SalI |
| | SEQ ID NO: 55 | SEQ ID NO: 53 | overlap extension | |
| | | | PCR | |
| ⁵³Nₜᵣₚ-SEC62 | PTP193 | PTP188 | ⁵³Nₜᵣₚ/SEC62, | BglII/SalI |
| | SEQ ID NO: 55 | SEQ ID NO: 53 | overlap extension | |
| | | | PCR | |
| ¹⁸⁷Nₜᵣₚ-SEC62 | PTP193 | PTP188 | ¹⁸⁷Nₜᵣₚ/SEC62, | BglII/SalI |
| | SEQ ID NO: 55 | SEQ ID NO: 53 | overlap extension | |
| | | | PCR | |
| ^{204b}Nₜᵣₚ-SEC62 | PTP193 | PTP188 | ^{204b}Nₜᵣₚ/SEC62, | BglII/SalI |
| | SEQ ID NO: 55 | SEQ ID NO: 53 | overlap extension | |
| | | | PCR | |

Primers used for Cₜᵣₚ-constructs (cf. attached sequence listing for details):

| | | | | cloning |
|---|---|---|---|---|
| construct | 5′-primer | 3′-primer | template(s) | sites |
| ⁴⁴Cₜᵣₚ-His | PTP155 | PTP194 | split-Trp⁴⁴ | — |
| | SEQ ID NO: 41 | SEQ ID NO: 56 | | |
| ⁴⁴Cₜᵣₚ-HA | PTP155 | PTP198 | split-Trp⁴⁴ | — |
| | SEQ ID NO: 41 | SEQ ID NO: 58 | | |
| ⁵³Cₜᵣₚ-His | PTP171 | PTP194 | split-Trp⁵³ | — |
| | SEQ ID NO: 44 | SEQ ID NO: 56 | | |
| ¹⁸⁷Cₜᵣₚ-His | PTP173 | PTP194 | split-Trp¹⁸⁷ | — |
| | SEQ ID NO: 46 | SEQ ID NO: 56 | | |
| ^{204b}Cₜᵣₚ-His | PTP175 | PTP194 | assembly PCR with | — |
| | SEQ ID NO: 48 | SEQ ID NO: 56 | primers PTP175, | |
| | | | PTP176, PTP179, | |
| | | | PTP180, PTP191, | |
| | | | PTP192 | |
| SEC63 | PTP189 | PTP154 | EGY48 yeast | — |
| | SEQ ID NO: 54 | SEQ ID NO: 40 | genomic DNA | |
| STE14 | PTP195 | PTP157 | EGY48 yeast | — |
| | SEQ ID NO: 57 | SEQ ID NO: 42 | genomic DNA | |
| SEC63-⁴⁴Cₜᵣₚ-His | PTP189 | PTP194 | SEC63/⁴⁴Cₜᵣₚ-His, | BglII/SalI |
| | SEQ ID NO: 54 | SEQ ID NO: 56 | overlap extension | |
| | | | PCR | |
| SEC63-⁵³Cₜᵣₚ-His | PTP189 | PTP194 | SEC63/⁵³Cₜᵣₚ-His, | BglII/SalI |
| | SEQ ID NO: 54 | SEQ ID NO: 56 | overlap extension | |
| | | | PCR | |
| SEC63-¹⁸⁷Cₜᵣₚ-His | PTP189 | PTP194 | SEC63/¹⁸⁷Cₜᵣₚ-His, | BglII/SalI |
| | SEQ ID NO: 54 | SEQ ID NO: 56 | overlap extension | |
| | | | PCR | |
| SEC63-^{204b}Cₜᵣₚ-His | PTP189 | PTP194 | SEC63/^{204b}Cₜᵣₚ-His, | BglII/SalI |
| | SEQ ID NO: 54 | SEQ ID NO: 56 | overlap extension | |
| | | | PCR | |
| STE14-⁴⁴Cₜᵣₚ-His | PTP189 | PTP194 | STE14/⁴⁴Cₜᵣₚ-His, | BglII/SalI |
| | SEQ ID NO: 54 | SEQ ID NO: 56 | overlap extension | |
| | | | PCR | |
| STE14-¹⁸⁷Cₜᵣₚ-His | PTP195 | PTP194 | STE14/¹⁸⁷Cₜᵣₚ-His, | BglII/SalI |
| | SEQ ID NO: 57 | SEQ ID NO: 56 | overlap extension | |
| | | | PCR | |
| SEC63-⁴⁴Cₜᵣₚ | PTP189 | PTP198 | SEC63-⁴⁴Cₜᵣₚ-His | BglII/SalI |
| | SEQ ID NO: 54 | SEQ ID NO: 58 | | |
| SEC63-⁵³Cₜᵣₚ | PTP189 | PTP198 | SEC63-⁵³Cₜᵣₚ-His | BglII/SalI |
| | SEQ ID NO: 54 | SEQ ID NO: 58 | | |
| SEC63-¹⁸⁷Cₜᵣₚ | PTP189 | PTP198 | SEC63-¹⁸⁷Cₜᵣₚ-His | BglII/SalI |
| | SEQ ID NO: 54 | SEQ ID NO: 58 | | |
| SEC63-^{204b}Cₜᵣₚ | PTP189 | PTP198 | SEC63-^{204b}Cₜᵣₚ-His | BglII/SalI |
| | SEQ ID NO: 54 | SEQ ID NO: 58 | | |
| STE14-⁴⁴Cₜᵣₚ | PTP195 | PTP198 | STE14-⁴⁴Cₜᵣₚ-HiS | BglII/SalI |
| | SEQ ID NO: 57 | SEQ ID NO: 58 | | |
| STE14-¹⁸⁷Cₜᵣₚ | PTP195 | PTP198 | STE14-¹⁸⁷Cₜᵣₚ-His | BglII/SalI |
| | SEQ ID NO: 57 | SEQ ID NO: 58 | | |

Primers used for zipper construction (cf. attached sequence listing for details):

| | |
|---|---|
| SEQ ID NO: 22: | PTP22 |
| SEQ ID NO: 23: | PTP23 |
| SEQ ID NO: 24: | PTP24 |
| SEQ ID NO: 25: | PTP28 |
| SEQ ID NO: 26: | PTP29 |
| SEQ ID NO: 27: | PTP100 |
| SEQ ID NO: 28: | PTP110 |
| SEQ ID NO: 29: | PTP111 |
| SEQ ID NO: 34: | PTP116 |
| SEQ ID NO: 35: | PTP117 |
| SEQ ID NO: 36: | PTP118 |
| SEQ ID NO: 37: | PTP119 |

Primers used for the copper promoter (cf. attached sequence listing for details):

| | |
|---|---|
| SEQ ID NO: 49: | PTP181 |
| SEQ ID NO: 50: | PTP182 |
| SEQ ID NO: 51: | PTP183 |
| SEQ ID NO: 52: | PTP184 |

Primers used for circular permutation of Trp1p (cf. attached sequence listing for details):

| | |
|---|---|
| SEQ ID NO: 30: | PTP112 |
| SEQ ID NO: 31: | PTP113 |
| SEQ ID NO: 32: | PTP114 |
| SEQ ID NO: 33: | PTP115 |

Primers used for homologous recombination (cf. attached sequence listing for details):

| | |
|---|---|
| SEQ ID NO: 63: | PTP107 |
| SEQ ID NO: 64: | PTP108 |
| SEQ ID NO: 38: | PTP120 |
| SEQ ID NO: 39: | PTP121 |

I claim:

1. A method for detecting an interaction between a first test peptide or protein or a fragment thereof, and a second test peptide or protein or a fragment thereof, the method comprising the steps of:
providing recombinant DNA sequences for use in securing expression of a first subdomain of a reporter protein and a complementary second subdomain of a reporter protein, wherein detectable activity of said reporter protein is restored, when said first subdomain and said complementary second subdomain are brought into close proximity, and wherein said first and said complementary second subdomain are not subdomains of one of the group of proteins consisting of transcriptional activators, ubiquitin, dihydrofolate reductase, β-lactamase, green fluorescent protein, β-galactosidase, inteins, cAMP cyclase, glycinamide ribonucleotide transformylase, aminoglycoside phosphotransferase, hygromycin B phosphotransferase, luciferase,
fusing an oligonucleotide or a gene encoding for a first test peptide or protein to the DNA sequence encoding for said first subdomain of the reporter protein, thereby creating a first DNA fusion sequence encoding for a fusion protein comprising said first subdomain of the reporter protein and said first test peptide or protein;
fusing an oligonucleotide or a gene encoding for a second test peptide or protein to the DNA sequence encoding for said complementary second subdomain of the reporter protein, thereby creating a second DNA fusion sequence encoding for a fusion protein comprising said complementary second subdomain of the reporter protein and said second test peptide or protein;
(co-) expressing said fusion protein comprising said first subdomain of the reporter protein and said first test peptide or protein, and said fusion protein comprising said second complementary subdomain of the reporter protein and said second test peptide or protein in a suitable prokaryotic or eukaryotic host cell;
screening and/or selecting for restoration of detectable activity of said reporter protein.

2. A method according to claim 1, wherein said DNA sequence encodes for a subdomain of a (β/α)₈₋barrel enzyme.

3. A method according to claim 1, wherein said DNA sequence is selected from the group consisting of:
(a) the DNA sequences SEQ ID NO 3, 5, 7, 9, 11, 13, 15, 17 or their complementary strands;
(b) DNA sequences which hybridize under stringent conditions to the protein coding regions of the DNA sequences defined in (a) or fragments thereof;
(c) DNA sequences which, but for the degeneracy of the genetic code, would hybridize to the DNA sequences defined in (a) or (b) and which sequences code for a polypeptide having the same amino acid sequence.

4. A method according to claim 1, wherein said DNA sequence is for use in securing expression in a prokaryotic or eukaryotic host cell of a polypeptide fusion product.

5. A method according to claim 1, wherein a library of oligonucleotides or DNA encoding for a set of first test peptides or proteins and/or a library of oligonucleotides or DNA encoding for a set of second test peptides or proteins are fused to said first subdomain of said reporter protein and/or said complementary second subdomain of said reporter protein, respectively.

6. A method according to claim 1, wherein said first test peptide or protein or a fragment thereof, and said second test peptide or protein or a fragment thereof, are peptides or proteins naturally occurring in compartments chosen from the group consisting of cellular membranes, the cytosol, the mitochondrium, the peroxisome and the lumen of the secretory path.

7. A method according to claim 1, wherein said interaction between a first test peptide or protein or a fragment thereof and a second test peptide or protein or fragment thereof is mediated by a chemical inducer of dimerization, which binds either covalently or non-covalently to both said test peptides or proteins or fragments thereof.

8. A method for detecting the interruption of an interaction between a first test peptide or protein or a fragment thereof, and a second test peptide or protein or a fragment thereof, the method comprising the steps of:
providing recombinant DNA sequences for use in securing expression of a first subdomain of a reporter protein and a complementary second subdomain of a reporter protein, wherein detectable activity of said reporter protein is restored, when said first subdomain and said complementary second subdomain are brought into close proximity, and wherein said first and said complementary second subdomain are not subdomains of one of the group of proteins consisting of transcriptional activators, ubiquitin, dihydrofolate reductase, β-lactamase, green fluorescent protein, β-galactosidase, inteins, cAMP cyclase, glycinamide ribonucleotide transformylase, aminoglycoside phosphotransferase, hygromycin B phosphotransferase, luciferase,
fusing an oligonucleotide or a gene encoding for a first test peptide or protein to the DNA sequence encoding for said first subdomain of the reporter protein, thereby creating a first DNA fusion sequence encoding for a fusion protein comprising said first subdomain of the reporter protein and said first test peptide or protein;
fusing an oligonucleotide or a gene encoding for a second test peptide or protein to the DNA sequence encoding for said complementary second subdomain of the reporter protein, thereby creating a second DNA fusion sequence encoding for a fusion protein comprising said complementary second subdomain of the reporter protein and said second test peptide or protein;
(co-) expressing said fusion protein comprising said first subdomain of the reporter protein and said first test peptide or protein, and said fusion protein comprising said second complementary subdomain of the reporter protein and said second test peptide or protein in a suitable prokaryotic or eukaryotic host cell;
screening and/or selecting for interruption of interaction of said first subdomain and said second subdomain under the influence of one or more test agents.
